(19) United States
(12) Patent Application Publication
(10) Pub. No.: US 2018/0185643 A1
(43) Pub. Date: Jul. 5, 2018

(54) CARDIAC HEALTH MONITORING SYSTEMS AND METHODS INVOLVING HYPERTENSION RELIEF DEVICE(S) AND/OR FEATURES

(71) Applicant: PHYSIOCUE, INC., Sunnyvale, CA (US)

(72) Inventors: Johnathan LEE, Sunnyvale, CA (US); Seon YI, San Jose, CA (US); Hugh Robert Sharkey, Redwood City, CA (US)

(21) Appl. No.: 15/828,273

(22) Filed: Nov. 30, 2017

Related U.S. Application Data
(60) Provisional application No. 62/428,460, filed on Nov. 30, 2016.

(51) Int. Cl. A61N 1/362; A61B 5/0408; A61B 5/021

(52) U.S. Cl. CPC A61N 1/3625; A61B 5/0408; A61B 5/0404; A61N 1/3621; A61B 5/02116

(57) ABSTRACT

Systems and methods involving hypertension monitoring and/or treatment device(s) are disclosed. According to implementations herein, various combination systems, devices and methods that provide therapy and/or monitoring capabilities of a person's blood pressure, incorporating a PPG/ECG integrated sensor within the handle of a hypothermia therapy device for persons with hypertension, are provided.

### CROSS REFERENCE TO RELATED APPLICATIONS

This application claims benefit/priority of U.S. provisional patent application No. 62/428,460, filed Nov. 30, 2016, which is incorporated herein by reference in entirety.

### BACKGROUND

### Field

The disclosed technology relates generally to health monitoring and treatment, and, more specifically, to implementations involving hypertension monitoring and/or treatment device(s) and methods.

### Description of the Related Information

The following discloses cardiac health monitoring systems and methods, aspects of which may bear relation to and/or involve features consonant with existing hypertension treatment devices, such as those of U.S. Pat. No. 7,713,295, issued May 11, 2010. The hypertension treatment device described in the above patent provides a treatment via thermal stimulation of baroreceptors located at the carotid sinus located in the neck region of a human body. To make treatment more effective and comprehensive, however, inclusion of heart activity monitoring systems, components, features and/or functionality within or associated with a device involve or yield further innovations, as well as improvements such as before-and-after heart activity changes, such as those resulting from treatment performed by the present device, and other novel aspects, outputs and/or results.

### Overview of Various Aspects of the Disclosed Technology

Since Blood Pressure (BP) is a measure on cardiovascular health condition, it will be helpful for understanding individual cardiovascular health status if other heart-related measures, such as Heart Rate (HR), Heart Rate Velocity (HRV), Electrocardiographic (ECG), and related abnormalities, can be monitored.

The most common method for acquiring blood pressure readings today involve an inflatable cuff type device either in conjunction with stethoscope auscultation of the arteries distal to the inflated bladder cuff (sphygmomanometers) or by sensors internal to the inflatable cuff that capture the bruit created by occluding, and then slowly release the tourniquet around the arteries of the upper arm. Due to the cumbersome method of applying the inflatable cuff appropriately to insure an accurate reading of these methods, conventional cuff-type blood pressure sphygmomanometers and digital blood pressure monitors are inherently awkward in providing a facile measure of blood pressure representative of cardiac pumping activity.

New methods for collecting blood pressure and several other physiologic parameters have been pursued using various sensors and algorithms that can analyze electocardiographic activity in combination with spectrographic readings of the variation vascular capillary color in the extremities, obviating the need for occlusive bladder type devices with devices that can be applied to the wrist and fingers, however these approaches have had varying success. Wrist and mobile devices that utilize PPG and/or ECG sensors have technical limitations. Devices that use a PPG sensor need a higher power light source in order to capture physiological data from the human body. Further, wrist and mobile devices are limited by their size and are unable to increase the battery power sufficiently. Additionally, the devices must come in direct contact with the skin without a cap between the skin and sensors; otherwise, the index would decrease the accuracy of the recorded data.

Implementations herein may include and/or involve an integrated system of a thermal module for hypothermic stimulation and a ECG/PPG (Photoplethysmography) module or sensor set for measuring BP, HR, HRV, and other cardiovascular health indices, achieving innovative implementations and functionality that far surpass the performance and utility of current physiologic monitoring systems, devices and/or methods.

According to some implementations herein, various combination devices that provide therapy and/or monitoring capabilities of a person's blood pressure, incorporating a PPG/ECG integrated sensor within the handle of a hypothermia therapy device for persons with hypertension, are provided. In certain aspects, after obtaining a pre-treatment blood pressure reading, devices herein may be placed against the neck over the carotid artery to effect stimulation of the carotid baroreceptors of the user, which triggers an autonomic nervous system response lowering blood pressure and heart rate. The blood pressure may then again be recorded, following treatment, to observe the results. Further, various monitoring functionality may be utilized, such as features involving the capability of uploading the measured physiologic data to a smart phone, other computing devices, etc. for viewing and archiving of the data, and for subsequent transmittal to the cloud or caregiver to manage the user's treatment regimen.

In addition, various implementations, systems, devices, and methods consistent with the present innovations may include and/or involve a wireless communication interface for transmitting measured data to smartphone, other mobile device, PC or other device or location so that the data can be displayed, stored, processed, and also transmitted to doctors, medical facilities such as hospitals and clinics, as well as other third parties or entities.

### BRIEF DESCRIPTION OF THE DRAWINGS

Various embodiments of the invention are disclosed in the following detailed description and the accompanying drawings

FIG. 1A illustrates an abstracted/block diagram of one exemplary device configuration including cardiac sensor features and functionality, consistent with one or more aspects of the innovations herein.

FIG. 1B-1D Illustrates views of an exemplary hypertension therapy device including aspects such as bio-sensor blood pressure monitoring components, electrodes and/or associated features, consistent with one or more aspects of the innovations herein.

FIG. 1E is an Illustration of correct and incorrect use of bio-sensor electrodes of an exemplary device, consistent with one or more aspects of the innovations herein.

FIG. 2 illustrates a block/flow diagram displaying the process by which the biosensor operates, such as in conjunction with a hypertension treatment device, consistent with one or more aspects of the innovations herein.

FIG. 3A illustrates an exemplary combined sensor set, including an ECG electrode/sensor and a PPG sensor, consistent with one or more aspects of the innovations herein.

FIG. 3B illustrates another exemplary bio-sensor and electrode configuration, including a PPG sensor, an ECG electrode/sensor, an optical/photo sensor and/or an LED, consistent with one or more aspects of the innovations herein.

FIG. 3C-3E illustrates an exemplary ECG sensor electrode in front, top, and side views, consistent with one or more aspects of the innovations herein.

FIG. 3F illustrates a diagram of an exemplary ECG and PPG sensor and electrode set/arrangement, in side view, consistent with one or more aspects of the innovations herein.

FIG. 4 illustrates a representative system configuration, including and/or involving exemplary ECG and PPG sensors sub-systems for measuring blood pressure, consistent with one or more aspects of the innovations herein.

FIG. 5 illustrates an exemplary ECG unit, consistent with one or more aspects of the innovations herein.

FIG. 6 illustrates one implementation of exemplary ECG signal processing, e.g. for detecting abnormalities, consistent with one or more aspects of the innovations herein.

FIG. 7 illustrates representative waveforms and exemplary parameters from ECG and PPG sensors/readings, consistent with one or more aspects of the innovations herein.

FIG. 8 illustrates an exemplary processing/flow diagram, e.g. for obtaining BP from ECG and PPG, consistent with one or more aspects of the innovations herein.

FIG. 9 illustrates various illustrative parameters of an exemplary ECG signal, consistent with one or more aspects of the innovations herein.

FIG. 10 illustrates various elements of an exemplary system and related transmission features, such as associated with the mobile applications within the mobile environment(s), consistent with one or more aspects of the innovations herein.

FIG. 11 illustrates an exemplary system including an application associated with a mobile device and involving mobile environment features, consistent with one or more aspects of the innovations herein.

FIG. 12 illustrates an exemplary system including an application shown in use with a mobile device and involving mobile environment features, consistent with one or more aspects of the innovations herein.

FIGS. 13A-13F illustrate exemplary innovations and associated mobile UI (user interface) aspects associated with representative implementations, including mobile device/environment innovations, consistent with one or more aspects of the innovations herein.

FIGS. 14A-14C and 15 illustrate exploded/layout view of exemplary devices and associated thermal, electronic and other components, consistent with one or more aspects of the innovations herein.

FIGS. 16A-16B illustrate exemplary views of the treatment tip, which may be a thermal tip, of an illustrative device, consistent with one or more aspects of the innovations herein.

FIG. 17A-17C illustrate layout positions of bio-sensor (ECG/PPG) and electrodes on steering wheels, consistent with one or more aspects of the innovations herein.

FIG. 18A-18B illustrate layouts of the cardiovascular indices with data such as human temperature, heart rate, blood pressure data and other physiological states in the automobile control display and/or main display panels, consistent with one or more aspects of the innovations herein.

### DETAILED DESCRIPTION OF ILLUSTRATIVE IMPLEMENTATIONS

Hypertension is a serious medical condition that is common among large portion of the population today. Estimated to be nearly 46% of American according to the new American Heart Association guidelines published in 2017. This condition is normally classified as being “high blood pressure” and is a result of blood flowing through blood vessels with a greater force than thought to be normal.

Increased blood flow within the human body can result from a number of factors: diabetes, obesity, smoking, lack of physical activity, and aging. These factors lead to a buildup of plaque and a stiffening of the walls in the arteries that cause an increase in blood pressure requiring the heart to work harder to maintain constant blood flow within the body. The greater pressure causes a strain on a subject's heart and results in further damage to blood vessels, as well as other problems such as myocardial infarction, kidney failure and stroke, which can lead to death.

A device previously disclosed in U.S. Pat. No. 7,713,295 implements the use of hypothermia therapy device to reduce hypertensive conditions through noninvasive means. Here, a temperature controlled hypothermic end of a more rudimentary shape is applied to the carotid sinus of a patient for a designated duration. This application of low temperature stimulation induces a baroreflex activation within the human body and can reduce the blood pressure of subject as a result.

According one or more of present implementations, the integration of advanced biosensor device(s) and/or sensors in conjunction with the hypothermic therapy device disclosed herein may be utilized to provide feedback innovatively to a user, such as feedback regarding the effectiveness of treatment(s) aimed at reducing hypertension. Systems and methods herein allow the user to monitor and record the progress of his or her blood pressure therapy and similar biological conditions as a result of utilizing the device and associated features and functionality. Along with the ability to monitor and track the effectiveness of the treatment, certain implementations also enable the user to easily share and analyze the data with family members, medical/health professionals, and/or other third parties or entities.

Various innovative systems and methods herein utilize new configurations of hardware, such as specialized PPG and ECG sensor sets, some of which having a concave design and/or be combined with software involved in the capture and analysis of the physiologic data. The concave sensor designs herein avoid ambient light interference with the specialized PPG receiver and interrelated ECG sensor capture features of the physiologic parameters. These innovative combinations are able to efficaciously filter human and mechanical noise and directly deliver the physiological data to the device. Additionally, one or more aspects of the disclosed technology may include an illumination source utilizing green light versus the more common red light illumination employed in other devices. New orientations of the light (illumination) component of the PPG sensor function, and novel algorithms processing the data from the novel hardware configuration provide a highly-accurate measures of blood pressure as compared to other technologies using PPG and ECG technologies in their attempts to provide meaningful, accurate information. Table 1 illustrates blood pressure data from one existing/competitor product, a cuff BP monitor data, versus the disclosed PPG/ECG sensor data and information resulting from the innovations herein. As shown here and in other tests, the systolic and diastolic data achieved from the presently-disclosed inventions are much closer to the actual results that those from such cuff style BP monitor.

TABLE 1
Blood pressure data from competitors cuff BP monitor
data versus the present PPG/ECG sensor-based implementations
PhysioCue Bio Sensor BP Measurement

| Omron 10 Series | | | PhysioCue | | | | | |
|---|---|---|---|---|---|---|---|---|
| BP Monitor | | | 2.0 Bio-Sensor | | | Percent Error | | |
| | Dias- | Heart | | | Heart | Sys- | Dias- | Heart |
| Systolic | tolic | Rate | Systolic | Diastolic | Rate | tolic | tolic | Rate |
| 120 | 80 | 77 | 121 | 72 | 76 | 0.8 | 10.0 | 1.3 |
| 131 | 85 | 76 | 131 | 81 | 76 | 0.0 | 4.7 | 0.0 |
| 125 | 87 | 74 | 126 | 81 | 72 | 0.8 | 6.9 | 2.7 |
| 125 | 81 | 71 | 128 | 82 | 73 | 2.4 | 1.2 | 2.8 |
| 117 | 75 | 66 | 117 | 70 | 69 | 0.0 | 6.7 | 4.5 |
| 117 | 74 | 75 | 116 | 65 | 77 | 0.9 | 12.2 | 2.7 |
| 168 | 92 | 64 | 172 | 104 | 60 | 2.4 | 13.0 | 6.3 |
| 116 | 73 | 78 | 116 | 71 | 86 | 0.0 | 2.7 | 10.3 |
| 129 | 80 | 69 | 120 | 82 | 70 | 7.0 | 2.5 | 1.4 |
| 123 | 80 | 70 | 121 | 76 | 70 | 1.6 | 5.0 | 0.0 |
| 129 | 79 | 71 | 113 | 71 | 74 | 12.4 | 10.1 | 4.2 |
| 148 | 98 | 102 | 153 | 102 | 92 | 3.4 | 4.1 | 9.8 |
| 125 | 90 | 74 | 124 | 82 | 82 | 0.8 | 8.9 | 10.8 |
| 121 | 77 | 71 | 125 | 80 | 66 | 3.3 | 3.9 | 7.0 |
| 118 | 71 | 75 | 115 | 75 | 72 | 2.5 | 5.6 | 4.0 |
| 125 | 80 | 69 | 121 | 80 | 67 | 3.2 | 0.0 | 2.9 |
| 120 | 80 | 65 | 127 | 75 | 68 | 5.8 | 6.3 | 4.6 |
| 110 | 74 | 64 | 120 | 81 | 70 | 9.1 | 9.5 | 9.4 |
| 109 | 73 | 89 | 117 | 66 | 91 | 7.3 | 9.6 | 2.2 |
| 122 | 83 | 72 | 134 | 86 | 79 | 9.8 | 3.6 | 9.7 |
| 126 | 71 | 71 | 123 | 77 | 76 | 2.4 | 8.5 | 7.0 |
| 122 | 74 | 57 | 128 | 84 | 57 | 4.9 | 13.5 | 0.0 |

### General

Various innovations herein may be implemented in many ways such as an apparatus, a system, or a computer software in conjunction with proper sensors, a processor, and a storage medium. In this invention, such an implementation is referred to as a system. In general, the disclosed system may have different components such as a processor, circuitry or a storage medium in order to be implemented within an existing device, and those components to process data are referred to as “processor”.

The following description of one or more embodiments of the invention is provided with accompanying figures that show the principles of the invention. The scope of the invention is limited only by the claims and the invention contains numerous alternatives, modifications, and equivalents.

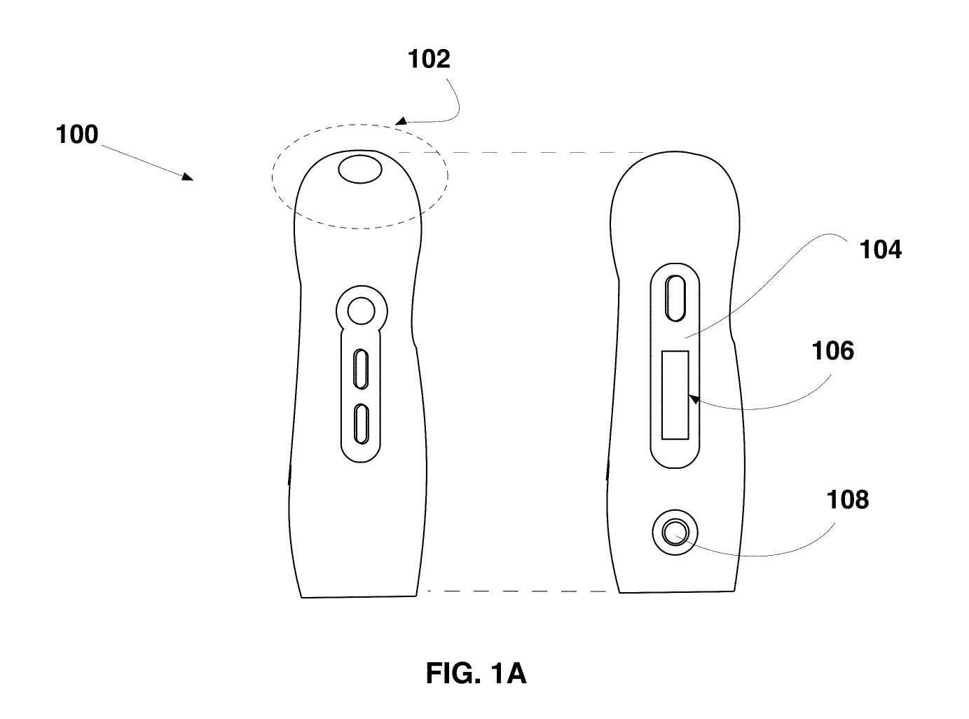

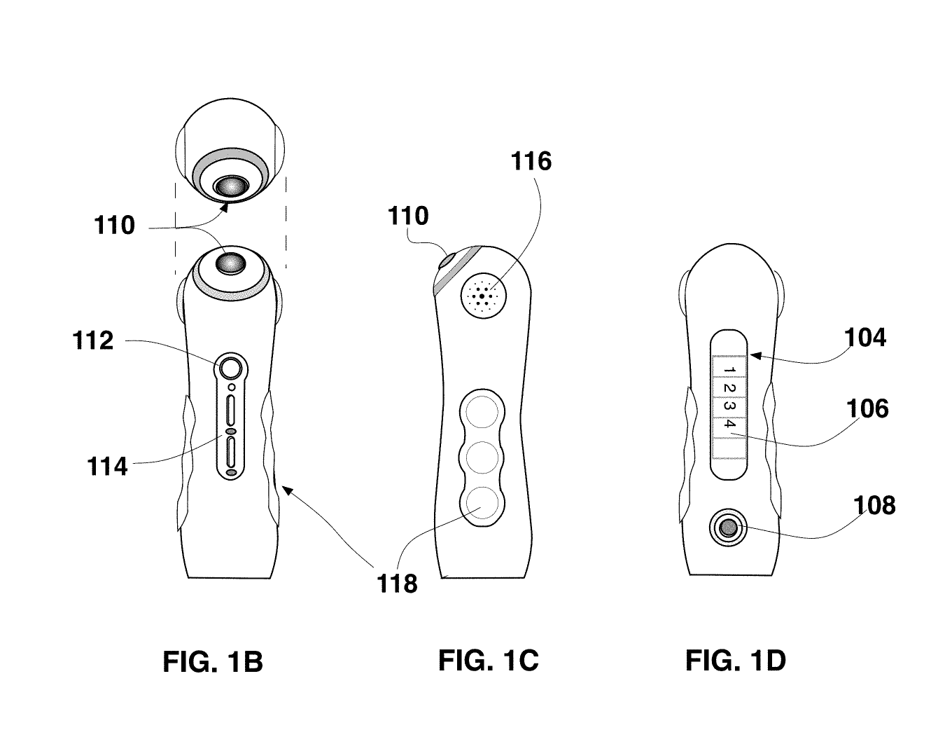

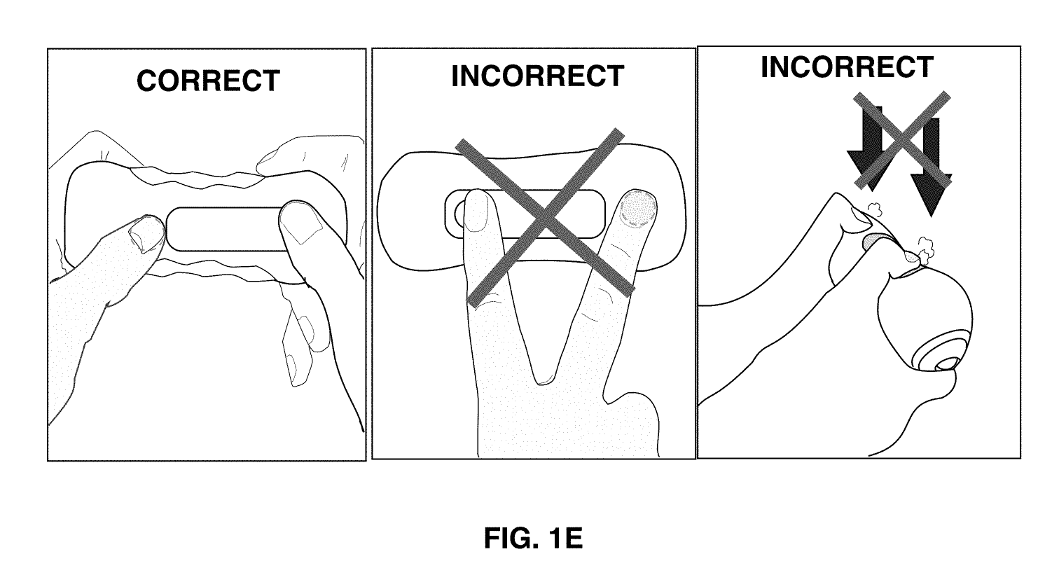

FIG. 1A illustrates an abstracted/block diagram of one exemplary device configuration with cardiac sensor features and functionality, consistent with one or more aspects of the innovations herein. Referring to FIG. 1A, a hypertension treatment device 100 is shown including a thermal portion 102, such as a tip, a first electrode/sensor 104, a display 106, and a second electrode/sensor 108. In the illustrative device shown, the first electrode/sensor 104 may comprise a first ECG (electrocardiographic) sensor, which in some implementations may serve as a reference electrode or node. Further, the second electrode/sensor 108 may comprise a combined electrode and sensor unit including a second ECG electrode and a PPG (photoplethysmorgraphic) sensor. In addition, the display may be configured to display physiologic information such as blood pressure, heart rate, other related information as shown in known heart monitoring devices, and other useful information, such as time, previous data readings and the like.

The thermal portion 102, which in some implementations may be shaped as part of a tip portion of the device 100, may provide pressure, cooling and/or other treatment features, such as set forth in more detail in the devices of U.S. Pat. No. 7,713,295, issued May 11, 2010, as well as published PCT patent applications WO2015/134394A1 and WO2015/134397A1 and their related U.S. counterparts, application Ser. No. 15/256,113, filed Sep. 2, 2016, published as US 2017/0049611 A1, and application Ser. No. 15/256,342, filed Sep. 2, 2016, published as US 2017/0056238 A1, all of which are incorporated herein by reference in entirety.

It is noted, here, that various implementations herein consistent with FIG. 1A may have the locations of such sensors fixed in the positions set forth in this and later drawings, and for certain implementations such positioning, size, shape and other physical characteristics form part of the innovations of these particular implementations. In other implementations, the positions and other physical characteristics of the sensors may be located in different regions of the device, and in some implementations, such as where no claim to such aspects is made, they may even be positioned in association with the device (e.g., attached, on associated portions, be other wired or wirelessly associated elements, be on a user's mobile device, etc.) rather than being unitary with, integrally embedded within, or the like with such device 100.

FIG. 1B-1D Illustrates views of a specific/exemplary hypertension therapy device including aspects such as bio-sensor blood pressure monitoring components, electrodes and/or associated features, consistent with one or more aspects of the innovations herein. Referring to FIGS. 1B-1D, the illustrated embodiment is shown with a particular hypothermia therapy tip 110 (the specific structure shown being deemed part of the innovation(s) in certain implementations), power button or switches and/or LED indicator(s) 112, treatment switches 114, such as for the hypothermia therapy tip (on the ‘top’ or side facing out in FIG. 1B) and/or the biosensors (bottom), one or more air exhausts or cooling vents 116 though some embodiments may not have such feature, a hand grip 118 that may be of a particular circumference, shape and/or pad structure. Further, as with the prior figure, an ECG electrode 104, a display 106, and a second PPG and ECG integrated electrode/sensor may be provided.

Additional features of the structure and precise geometric shape of various devices included within the ambit of the innovations herein are set forth in more detail in Appendix A, which shows various diagrams of such devices in dimension scale (angles, shape, etc.) consistent with certain implementations.

FIG. 1E is an Illustration of correct and incorrect use of bio-sensor electrodes of an exemplary device, consistent with one or more aspects of the innovations herein. Referring to FIG. 1E, correct usage is shown in the first panel, incorrect placement of two fingers of one hand (rather than a finger from each hand) is shown in the second panel, and improper use of pressing down on the sensors with force is shown in the third panel.

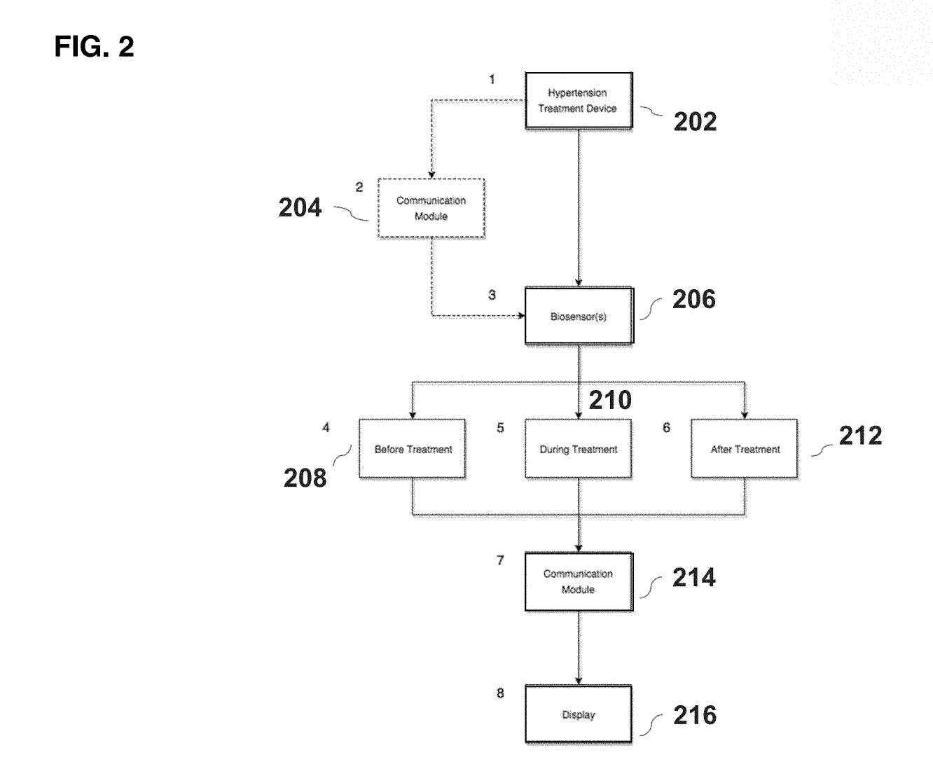

According to some implementations, systems or devices consistent with the innovations herein may also include and/or involve a biosensor for use in conjunction with a hypertension treatment device set forth in U.S. Pat. No. 7,713,295, as well as published PCT patent applications WO2015/134394A1 and WO2015/134397A1 and their related U.S. counterparts, application Ser. No. 15/256,113, filed Sep. 2, 2016, published as US 2017/0049611 A1, and application Ser. No. 15/256,342, filed Sep. 2, 2016, published as US 2017/0056238 A1, all of which are incorporated herein by reference in entirety. Such systems or products may be integrated into or used as a separate device in connectivity with said hypertension treatment device as a means of monitoring treatment efficiency and hypertensive conditions. In certain implementations, blood pressure measurements may be taken via such device(s) before, during, and after treatment as shown in FIG. 2.

FIG. 2 illustrates a block/flow diagram displaying the process by which the biosensor operates, such as in conjunction with a hypertension treatment device, consistent with one or more aspects of the innovations herein. According to the implementations illustrated, various functionality and pathways are shown, as taken e.g. by both the integrated and separate biosensors. In certain implementations, a separate biosensor may have one extra step of communicating with the hypertension treatment device before taking a measurement. Referring to FIG. 2, a hypertension treatment device 202 may include and/or involve a communication module 204 that interconnects the device with the biosensor(s) 206. In conjunction with such connectivity and communications, various processing, decision-making and/or information may be calculated (and, in some implementations, displayed) all of before treatment 208, during treatment 210, and/or after treatment 212. Such processing may then be combined or otherwise integrated with the data and results of the treatment, and provided back to the communication module 214 or other such processing module(s) for any final refinement or processing to configure the information for display 216.

### Electrodes/Sensors

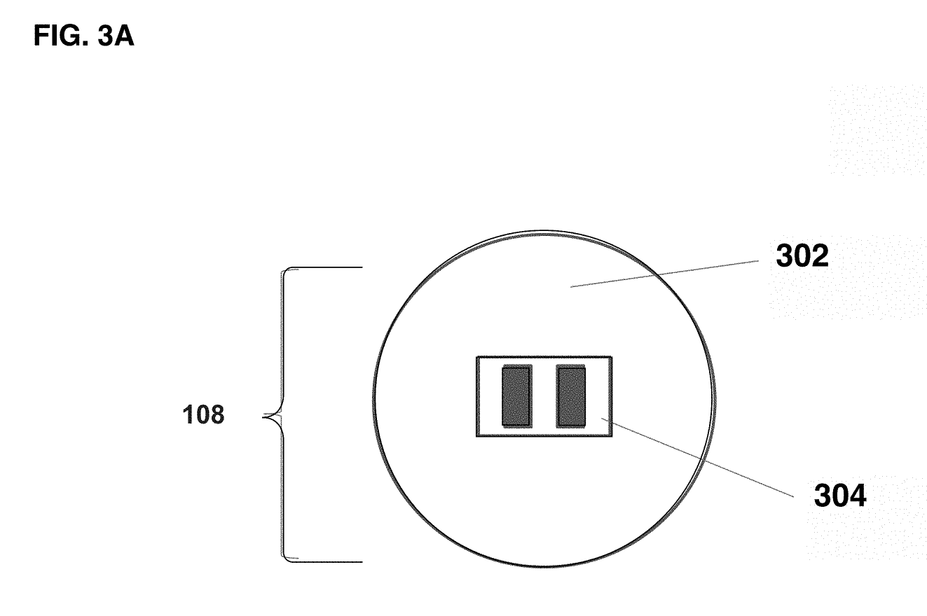

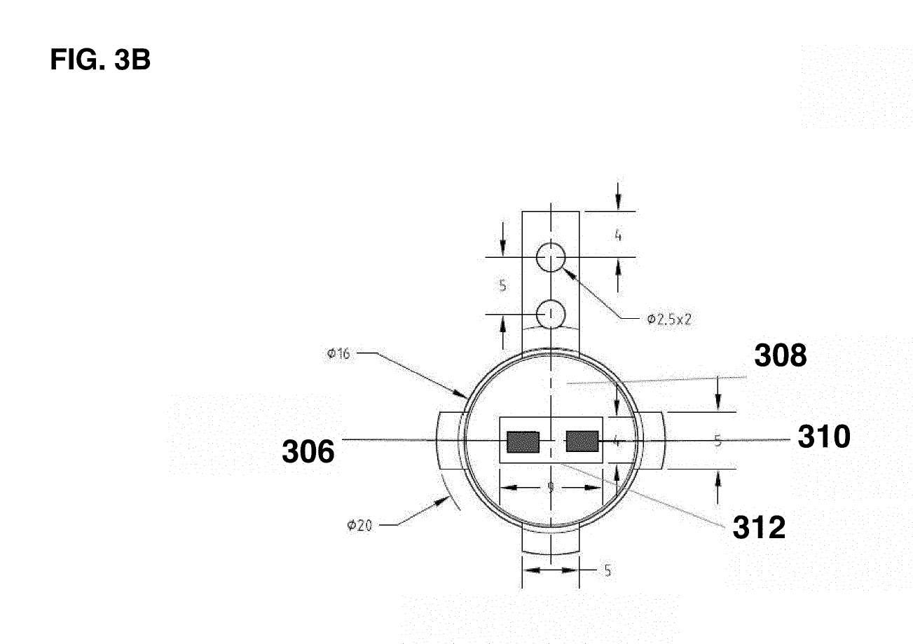

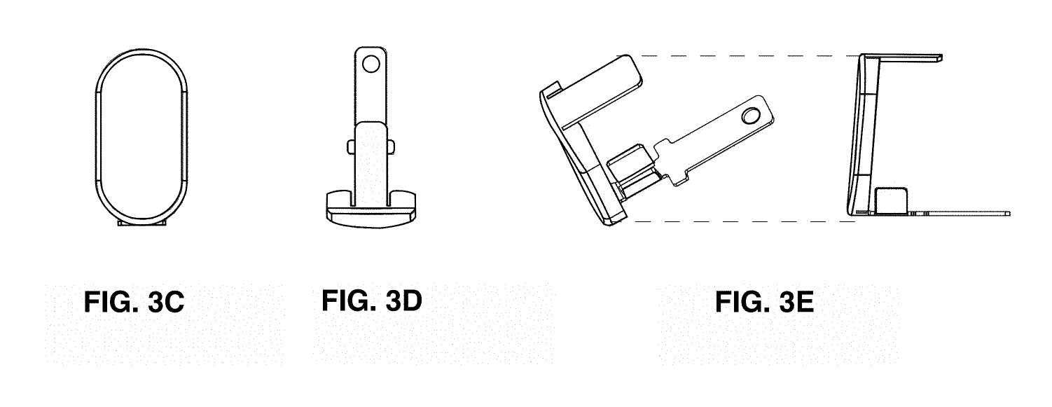

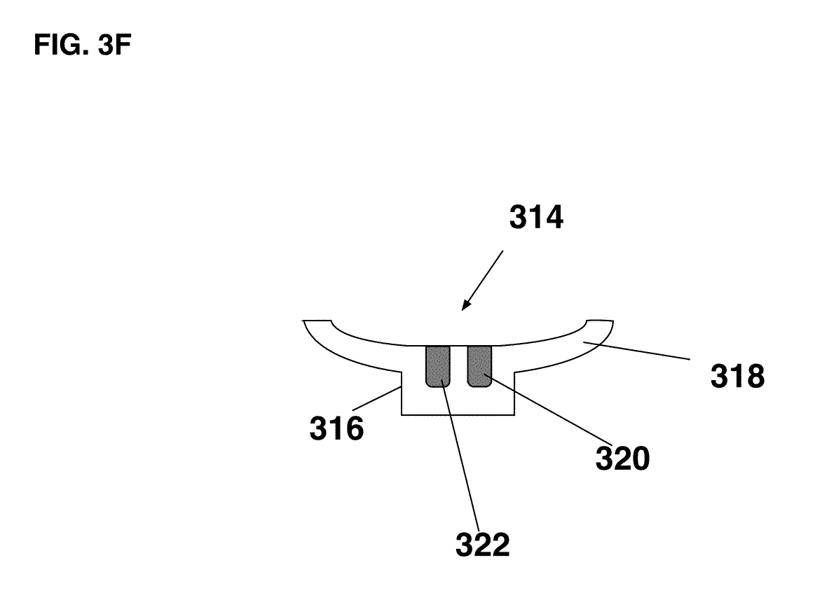

FIGS. 3A-3F illustrate various features and aspects of electrodes/sensors as may be present in various implementations of the innovations herein. FIG. 3A illustrates an exemplary combined sensor set 108, including an ECG electrode/sensor 302 and a PPG sensor 304, consistent with one or more aspects of the innovations herein. Referring to FIG. 3A, an abstraction of a top view of such electrode/sensor is shown, which may correspond to the second electrode/sensor as discussed above in connection with FIG. 1A. FIG. 3B illustrates further details of an exemplary bio-sensor and electrode configuration, including a PPG sensor 312, an ECG electrode/sensor 308, and photo-sensor 310 and/or light 306 (e.g., LED, etc.) components, consistent with one or more aspects of the innovations herein. Referring to FIG. 3B, details of the light 306 and photo-sensor 310 subcomponents of an exemplary PPG sensor are shown, along with various extensions of the metal ECG electrode extending above and to the sides of the combined electrode/sensor. FIG. 3C-3E illustrates an exemplary ECG sensor electrode in front FIG. 3C, top FIG. 3D, and orthogonal views FIG E, consistent with one or more aspects of the innovations herein. These figures provide illustrative detail of one exemplary first electrode/sensor 104, pertaining to the shape of the metal structure of this representative ECG electrode. FIG. 3F illustrates a diagram of an exemplary ECG and PPG sensor and electrode set/arrangement, in side view, consistent with one or more aspects of the innovations herein. Referring to FIG. 3F, additional side-view details of the combined electrode/sensor set are shown, showing a concave shape 314 and including the PPG sensor set 316 (with light/LED 322 and photo-sensor 320), and the ECG electrode 318, according to some embodiments.

### Integrated Device

Various systems and devices disclosed herein may be integrated into said hypertension treatment device to function as one complete device as shown in FIGS. 1A-1D. The integrated device can measure the blood pressure of a patient before, and after treatment by the hypertension treatment device. The user can measure his/her initial blood pressure while the hypertension treatment device reaches required conditions for treatment. Blood pressure can then be measured again after the treatment. The post treatment measurement requires a time delay before observation. This is due to the time required for the hypertension treatment to take effect. Implementations of such systems and integrated devices may have a timer to communicate with the biosensor device regarding the measurement of initial, during, and final blood pressures.

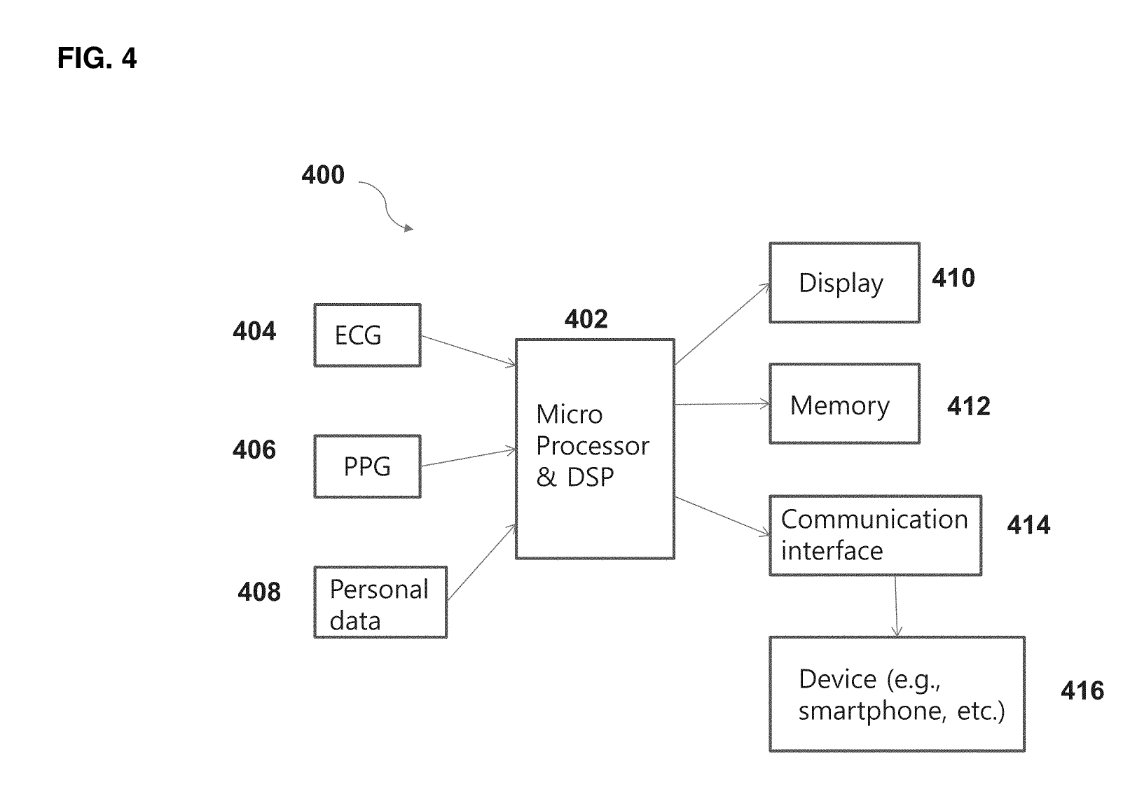

FIG. 4 illustrates a representative system 400 configuration, including and/or involving exemplary ECG and PPG sensors sub-systems for measuring blood pressure, consistent with one or more aspects of the innovations herein. Referring to FIG. 4, an exemplary system is shown comprising one or more processing components 402 such as a microprocessor and/or digital signal processing (DSP) module, an ECG sensor 404, a PPG sensor 406, information and/or data 408 such as personal data that may be generated by the device or come from or be derived from other sources, at least one display 410 which in some embodiments is integral with the device though is not necessarily (e.g., a display of another device may be utilized, etc.), one or more data stores 412 such as memory that may be integral with or external to the device, a communication interface 414, and/or (in certain embodiments) another device 416, such as a personal device, a mobile device, a mobile/cell phone, etc.

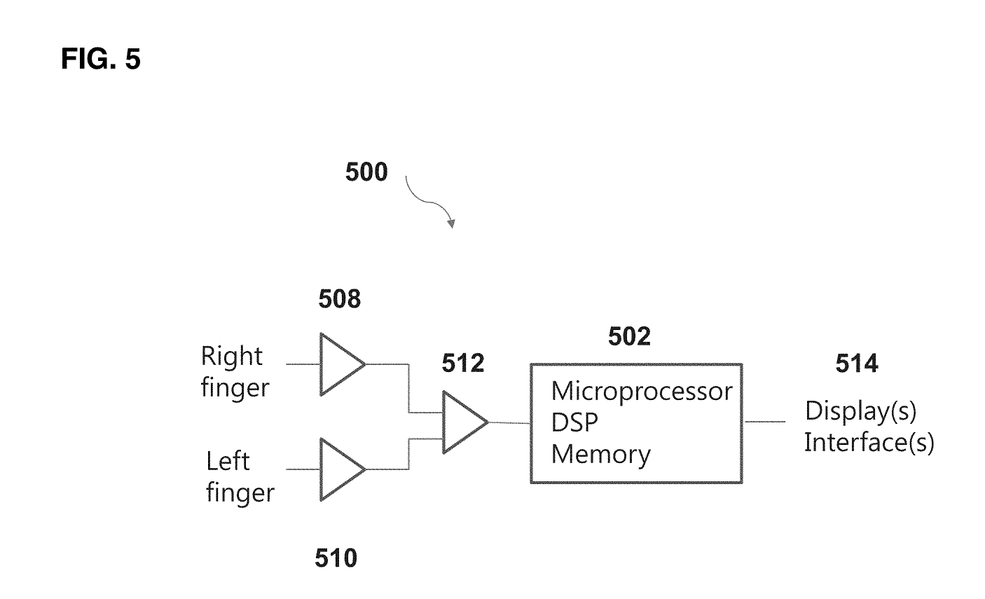

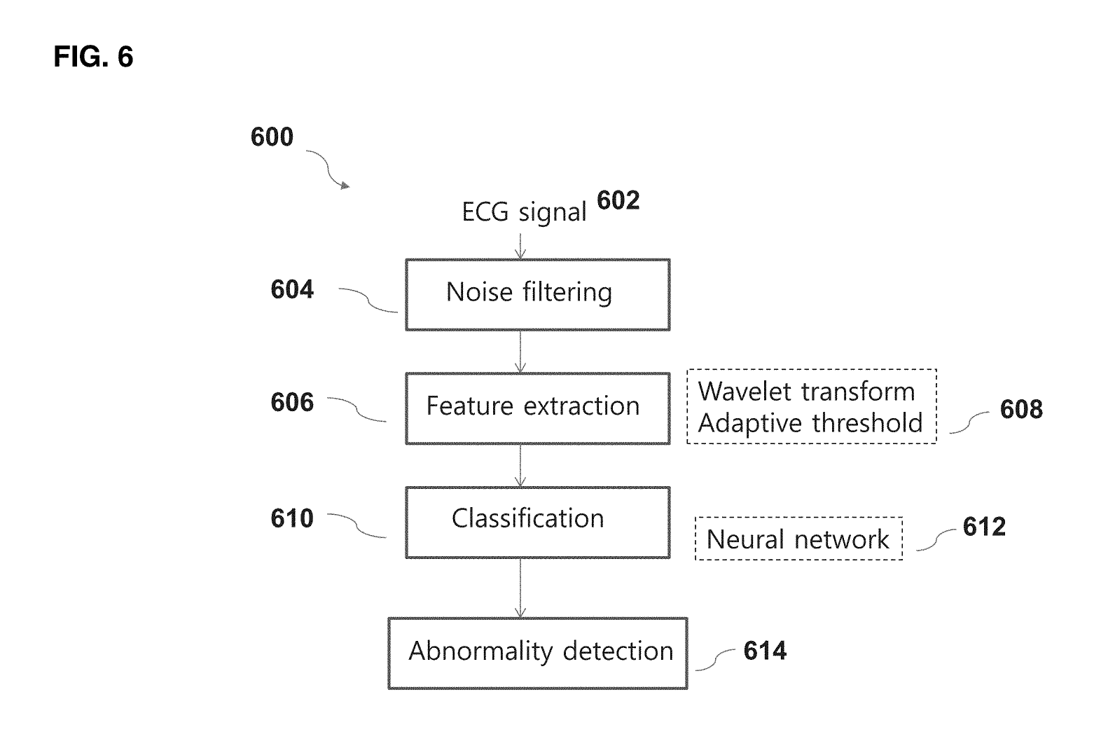

FIG. 5 illustrates an exemplary ECG unit, consistent with one or more aspects of the innovations herein. Referring to FIG. 5, the exemplary ECG unit shown may include a first sensor 508 for a user's right finger 504, a second sensor 510 for a user's left finger, a filtering module, element or system 512 that filters various environmental, air, light and/or human body noise components out of the signal(s) from the user, one or more computing/processing elements 502 such as a microprocessor, a digital signal processor (DSP), and/or a memory, and one or more display(s) and/or interface(s) 514. Such computing/processing element(s) 502 may be utilized, for example, to perform the ECG signal processing set forth in more detail in FIG. 6.

FIG. 6 illustrates one implementation of exemplary ECG signal processing 600, e.g. such as for detecting abnormalities, consistent with one or more aspects of the innovations herein. Referring to FIG. 6, implementations herein may process an ECG signal 602 including noise processing 604, performing feature extraction 606, performing classification 610, and/or one or more steps associated with abnormality detection 614. With regard to feature extraction 606, for example, implementations may perform wavelet transform and/or adaptive threshold 608 related processes to extract features, among other things. With regard to classification 610, implementations may utilize neural networks to classify the measured data, in addition to other techniques described herein and know in the field.

### Parallel Device

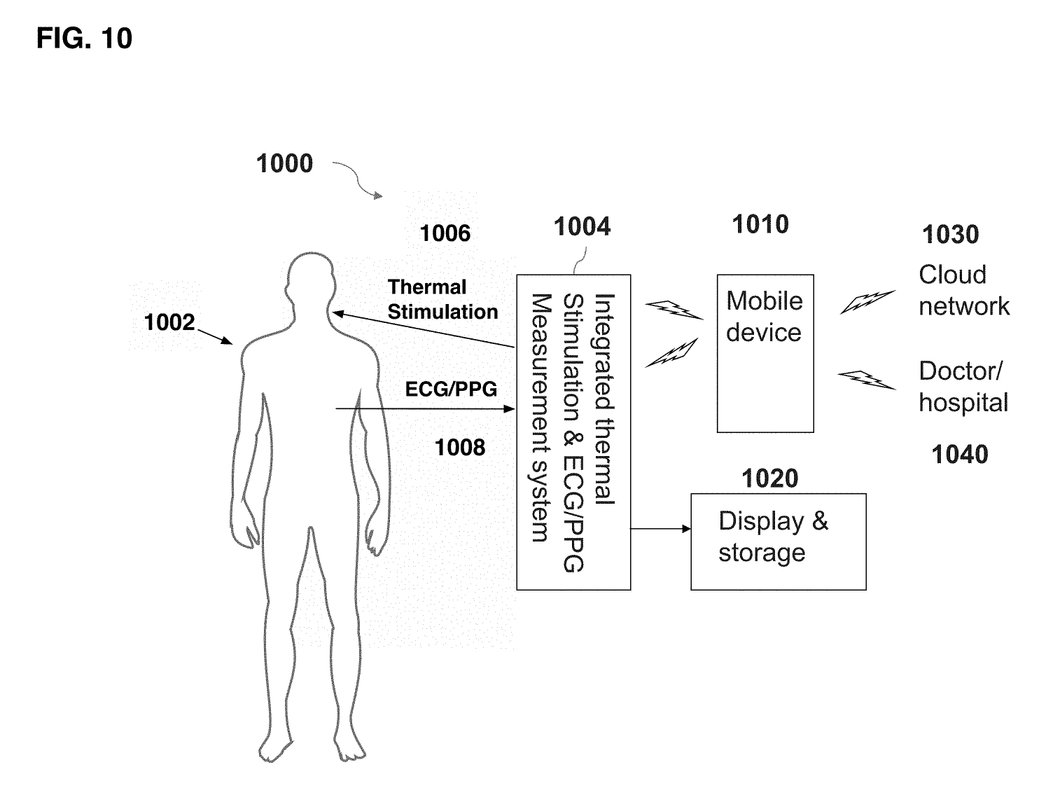

A device disclosed herein can be separate from the hypertension treatment device as shown in FIG. 10, described in more detail below. The device can be connected to the hypertension treatment device via a wireless communication module to ensure parallel functionality between both devices. The parallel device can measure the blood pressure of a patient before, during, and after treatment by the hypertension treatment device. The user can measure his/her initial blood pressure while the hypertension treatment device reaches required conditions for treatment. Blood pressure can then be measured during and after the treatment. The post treatment measurement requires a time delay before observation. This is due to the time required for the hypertension treatment to take effect. The parallel device can have communication with the hypertension treatment device regarding the proper times to take the initial, during, and final blood pressure measurements.

### Wireless Communication

A device disclosed herein can include a wireless communication module to possess the ability to transmit or receive data from one or more PCs, mobile phones, or of other mobile devices using the Cloud, Bluetooth, BLE, WiFi, ZigBee, RF, and other wireless network. In addition, measured and analyzed personal data can be stored in the cloud network, which only authorized users can upload and download data and analyzed results. All data in the cloud network are encrypted for personal privacy and security. The wireless communication module can allow the user to interface the hypertension treatment device with the biosensor or other devices to transmit information such as blood pressure and/or physiologic measurements. The wireless communication module can allow the parallel device to communicate with the hypertension treatment device regarding the proper times to take the initial and final blood pressure measurements.

### Data Logging

A device disclosed herein can include a data logging module to possess the ability to store information received from the biosensor as a means of monitoring treatment efficacy over extended periods of time. Once this information is logged, a communication module can be used to send the data to a display (i.e., a built-in display, phone, tablet, computer, etc.). The data logging module can help the user display the treatment effects as a result of the hypertension treatment device and share that information with family members and/or medical/health professionals.

Prior to the hypertension treatment device being applied to the patient, the biosensor device will be used to measure the patient's blood pressure and/or other physiologic measurements as a comparison point for during-treatment and post-treatment values.

### Steering Wheel

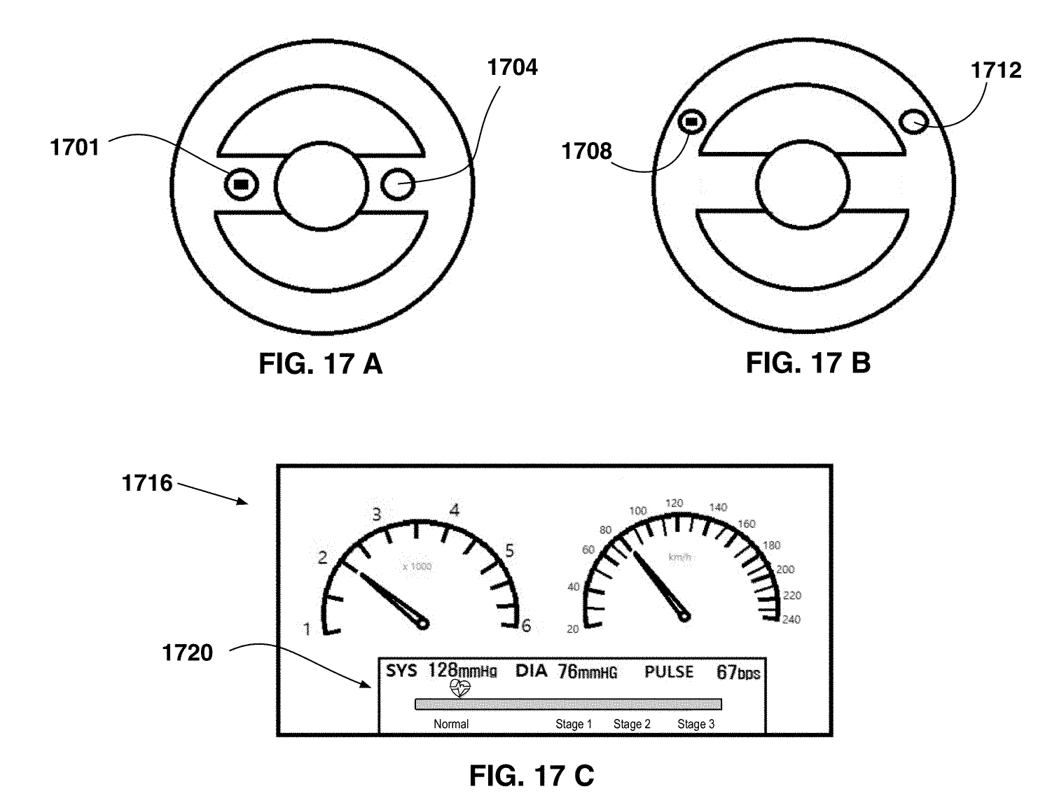

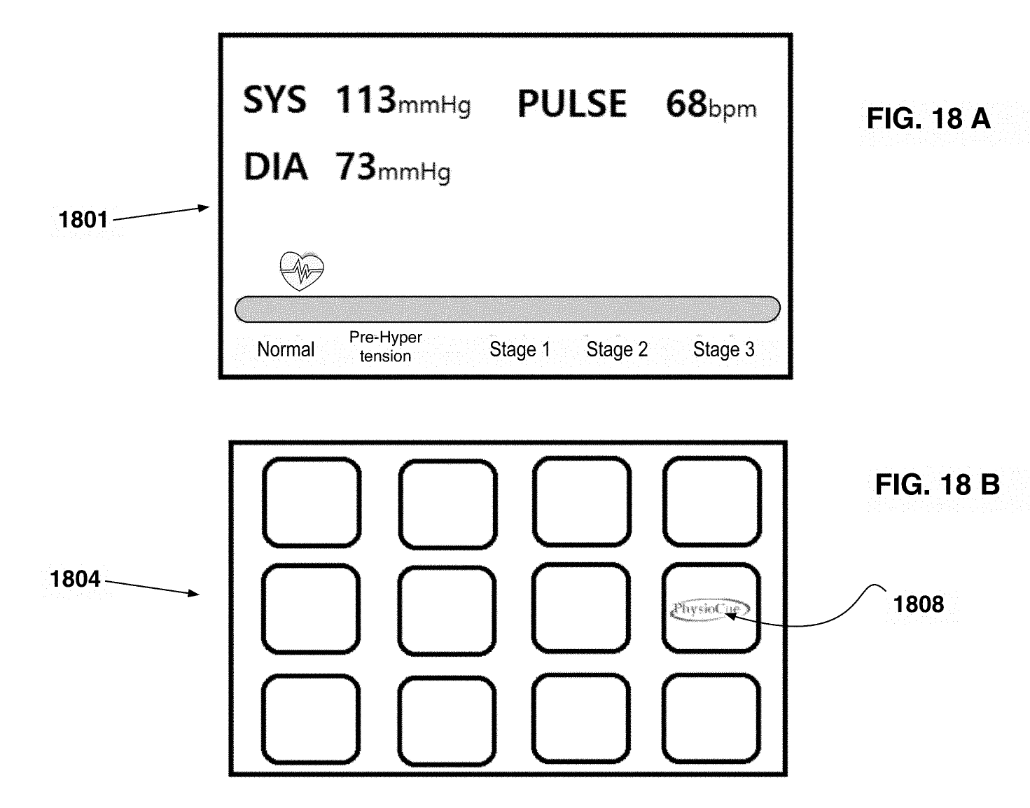

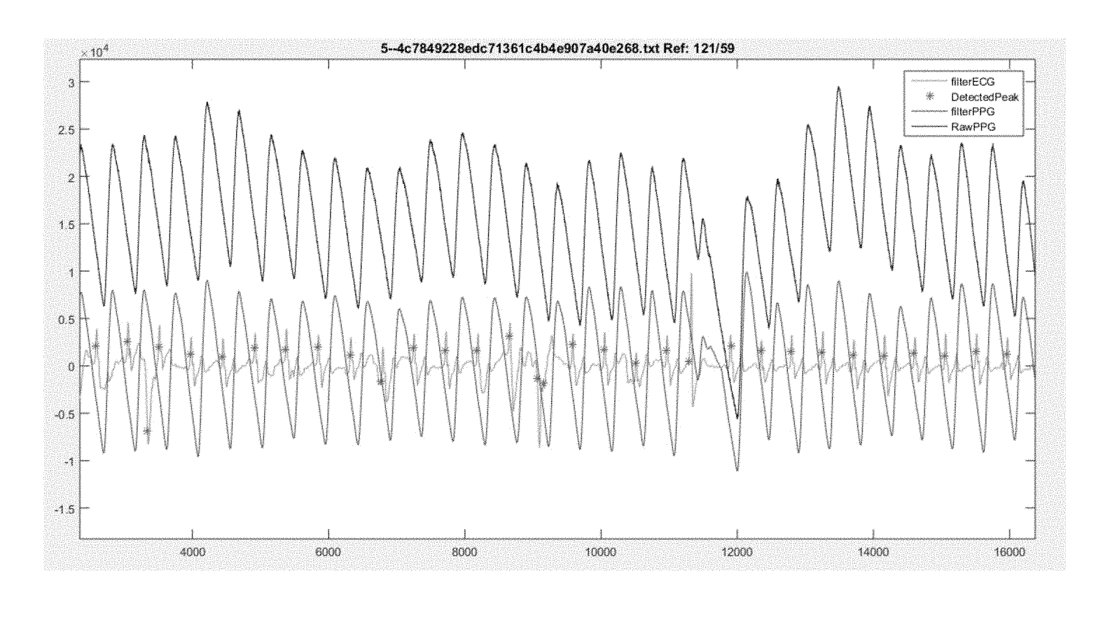

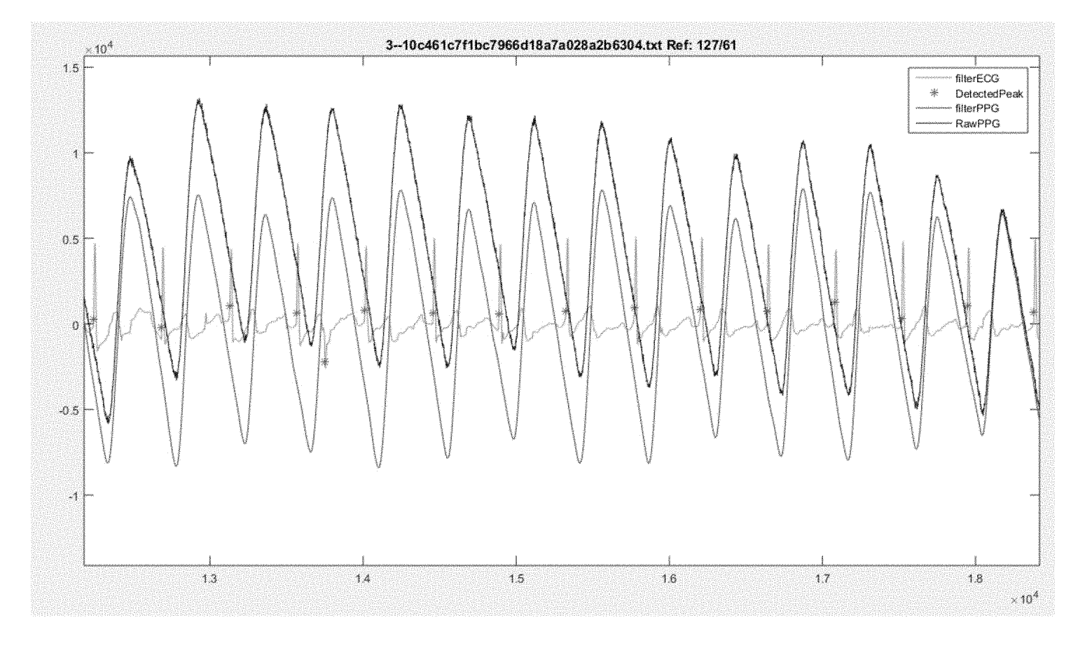

As discussed in more detail below in connection with FIGS. 17-18, embedded bio-sensors (PPG & ECG) may be placed inside on an automobile or other vehicles' steering wheel. By placing a person's thumbs on the sensors on the steering wheel before, during and/or after driving, the automobile's ECG/PPG signals will provide cardiovascular health indices and measure cardiovascular health data from drivers' and/or passengers' fingers. This is an efficient and comfortable interface that measures, records, and analyses cardiovascular health data. In further implementations, such data may be transmitted to the automobile (or other vehicle) control display and/or main automobile display panels. Such implementations are valuable, as a variety of present automotive innovation lies outside of the sole focus of driving.

### Overview of Certain Cardiovascular Health Monitoring Implementations

A fundamental non-invasive measure of cardiovascular activity is the ECG. Traditionally ECG signal can be recorded by the electrodes located on the chest, the wrists and the ankles Recently new ECG sensor technology is developed for measuring ECG from the fingertips or the wrist. However this technology requires a circuit across the body most often via hands or arms.

It is possible to compute many cardiovascular health indices from the ECG. HR, HRV, R-R interval, and even the respiratory rate can be calculated.

PPG can also be used to calculate HR, HRV and beat-to-beat interval. PPG sensor is generally located at the fingertip or earlobe. It however can be located on the skin over any blood vessel.

Blood pressure is an important measure of cardiovascular health status. Currently there are two common and non-invasive methods to measure arterial blood pressure. The Auscultatoric method requires an aneroid sphygmomanometer (an inflatable bladder placed around the upper arm typically connected to a mechanical pressure gauge) and a stethoscope to listen to the blood flow during inflation and deflation of the cuff in order to obtain blood pressure and is a widely accepted method in the clinical environment. The Oscillometric method also requires an inflatable bladder or arm cuff, but uses a pressure sensor located in the bladder to acquire the blood pressure reading. It is not uncommon to find digital oscillometric blood pressure monitors as well as sphygmomanometers in hospital and otherplaces.

Non-Invasive Blood Pressure Measurement with Biosensors

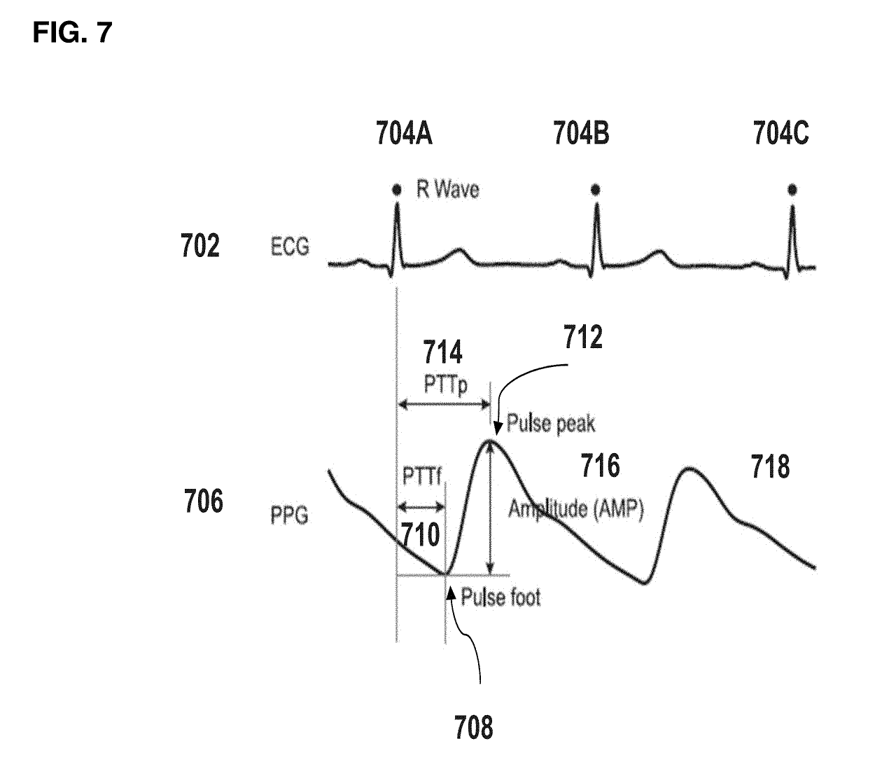

The circulatory system is, in principle, a hydraulic system, thus it means that one can monitor changes in the blood pressure in artery by obtaining on pulse wave velocity and the time delay of the pulses. The speed of the arterial pressure wave travels in known to be directly proportional to BP. The pulse wave velocity (PWV) can be measured using ECG and PPG signals. PTT (Pulse Transit Time), 1/PWV, is generally used to compute BP. PTT is defined as the time the pulse travels between two arterial sites within the same cardiac cycle. When both the ECG and PPG signal are recorded, the PTT is the time the R peak of the ECG and systolic peak of the PPG pulse, as seen, for example in FIG. 7.

FIG. 7 illustrates representative waveforms and exemplary parameters from ECG and PPG sensors/readings, consistent with one or more aspects of the innovations herein. Consistent with the innovations herein and as helpfully shown via reference to FIG. 7, measurement of one's blood pressure may be indirectly calculated from electrocardiography (ECG) 702 and photoplethysmorgprahy (PPG) 706 biometric signals. ECG measures electrical activities of the heart by using electrodes attached to a human skin. In this invention, both right and left hands are going to be in contact with electrodes in the sensor module. PPG measures volumetric changes of blood in vessel by using optical sensor(s) such as a combination of a light source (e.g., LED, etc.) and a photo-detector such as a photo-transistor (PT). Once ECG and PPG signals from the sensors are filtered and amplified properly, Post Transfer Time (PTT), the time interval between adjacent peak points 704/712 of ECG and PPG in the same cardiac cycle, can be calculated (see PTTp 714 in FIG. 7). PPG amplitude 716 and pulse foot 708 are also shown in FIG. 7, and a value for PTTf 710 may also be calculated and used. Although the process of depicting PTT appears straightforward, how PTT is defined and obtaining accurate series of PTTs via algorithms is one aspect to determining BP according to certain implementations. In many researches regarding non-invasive BP measurement, PTT values have been derived by fixing R-peck value of ECG as a reference point due to easy detection and correspondence to the systole of ventricles. However, there is an inherent, non-constant pulse delay between ECG and PPG signals presented in each cardiac cycle due to pulse being delayed depending on how far ECG and PPG sensors are located from the heart.

Once PTT values are obtained from ECG and PPG signals, PWV may be calculated by dividing PTT by the distance from the heart to the location of the sensor. BP is then applied to a linear model as below:

BP=a*PWV+b or

BP=c*PTT+d

Then calibration process is applied to the model to determine coefficients, a, b, c and d In an example case, it was shown that the systolic BP with PTT could be calculated by

Systolic BP=−0.69×PTT+228.59

In other example case, it's shown that the systolic BP and diastolic BP can be calculated with PWV.

Systolic BP=0.051×PWV+62.56 Diastolic BP=0.05×PWV+17.48

### HR, HRV, and Other Cardiovascular Health Indices

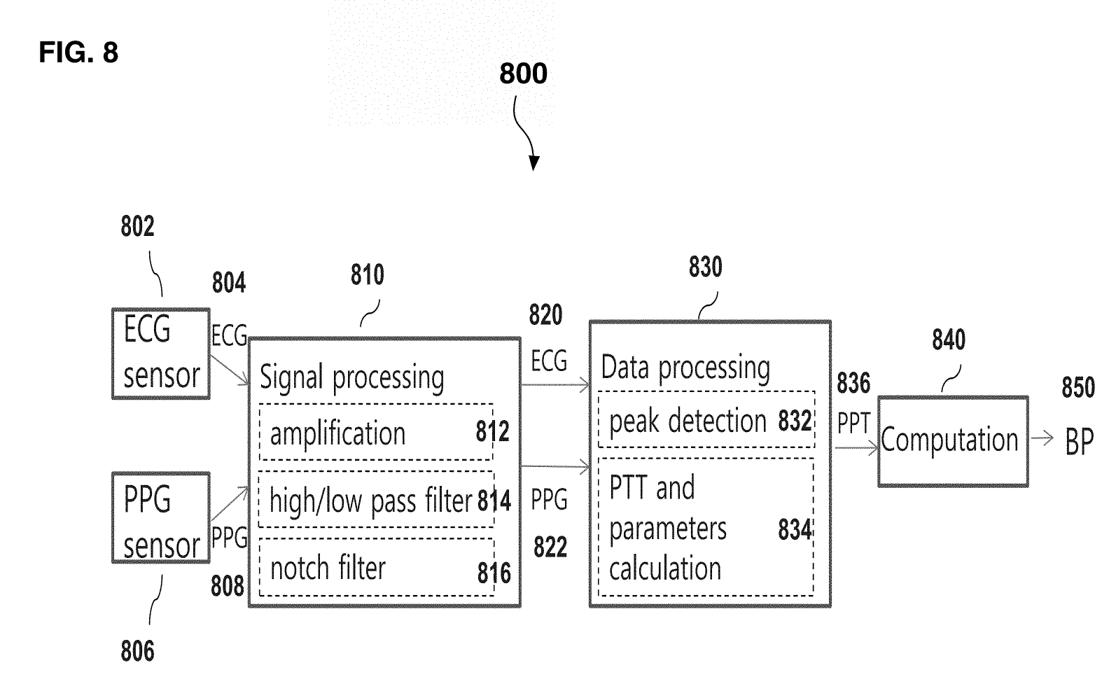

FIG. 8 illustrates an exemplary processing/flow diagram, e.g. for obtaining BP from ECG and PPG, consistent with one or more aspects of the innovations herein. Referring to the illustrative implementations of FIG. 8, the ECG sensor 802 and the PPG sensor 806 may provide the ECG signal 804 and the PPG signal, respectively, to one or more signal processing modules or components 810. Such signal processing may include, by way of example and not limitation, one or more of amplification 812, high and/or low pass filtering 814, and/or notch filtering 816. The filtered/clean ECG and PPG signals 820, 822 may then be provided to one or more data processing modules or components 830, which may further process the signals such as by performing peak detection and performing algorithms to provide the PTT and parameter calculations. In some implementations, a calculated PTT value 836 may then be provided to another computational unit 840 or otherwise be processed to provide final output(s) 850 such as a blood pressure measurement.

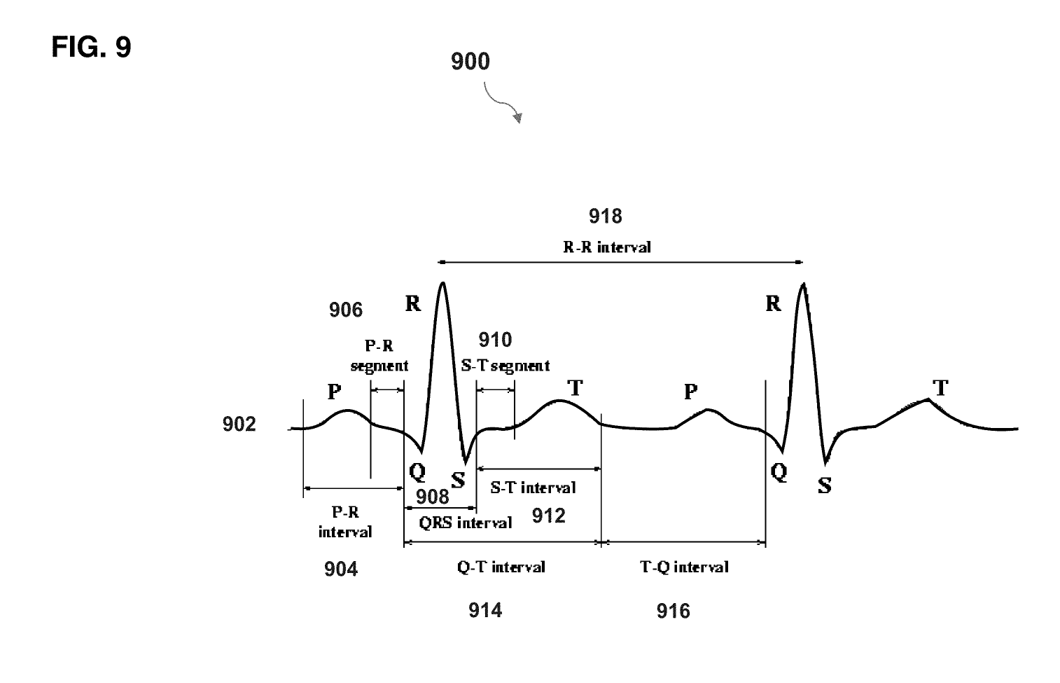

FIG. 9 illustrates various illustrative parameters of an exemplary ECG signal 902, consistent with one or more aspects of the innovations herein. Consistent with FIG. 9, an ECG signal is characterized by 5 peaks and valleys, named by P, Q, R, S and T waves, and various associated intervals and segments 904, 906, 908, 910, 912, 914, 916. The P wave represents the activation of the upper chamber of the heart. The QPS complex and T wave represent the excitation of the lower chamber of the heart, the ventricle. The Q-T interval 914 is normally less than 0.42 seconds, and the normal HR ranges from 60 to 100 BPM in rest state.

The QRS complex 908 is a prominent feature of the ECG signal, which is associated with cardiac health and implementations herein. Accurate detection of the QRS complex can form the basis of extraction of other features and parameters from the ECG signal. There are many known techniques for detecting the QRS complex from the ECG signal. Fast Fourier Transform (FFT), Discrete Fourier Transform (DFT), and Wavelet transforms are widely used for detecting QRS complex. Some techniques use amplitude, slope and threshold limit in addition to filters and mathematical functions. Several new techniques were developed based on Artificial Neural Network (ANN), fuzzy logic, and genetic algorithm. Combinations of the Wavelet transform, adaptive threshold, and neural network algorithms can be used to feature extraction and classification of the QRS complex. Monitoring R-R interval of the ECG signal makes possible to detect atrial fibrillation. Cardiac arrhythmia can also be detected based on the rhythm of the ECG.

Some cardiac abnormalities which can be characterized by ECG patterns are as follows;

| Cardiac abnormalities | ECG pattern |
|---|---|
| Atrial Fibrillation | Abnormal R-R interval |
| Dextrocardia | Inverted P wave |
| Tachycardia | R-R interval is shorter than 0.6 seconds |
| Bradycardia | R-R interval is longer than 1 second |
| Hyperkalemia | Tall T wave and absence of P wave |
| Hypercalcaemia | QRS interval is shorter than 0.1 seconds |
| Ventricular tachycardia | Irregular or fast ECG. |
| R-R intervals | |

There are many cardiovascular health indices which can be computed from the ECG signal processing; HR, HRV, respiratory rate (RR), heart age, and stress level.

A normal HR ranges between 60 and 90 BPM. If the HR drops below 50 bpm, it is referred to as Brachycardia. While, if the HR exceeds 100 bpm it is referred to as tachycardia. The average maximum HR with exertion of a normal person is computed as 220 minus the age of the person in years. For an example, an average maximum HR of 40 year old person will be 180 bpm.

HRV includes any indices; SDNN, rMSSD, LF, HF, LF/HF, etc. Normal values and ranges are as follows (HRV: European Heart Journal, 17, 354-381, 1996) SDNN (Standard deviation of NN intervals, ms): 50 (ranges 32˜93) rMSSD (Root mean square of successive differences between NN intervals, ms): 42 (ranges 9˜75) LF (Low frequency power, ms²): 519 (ranges 193˜1009) HF (High frequency power, ms²): 657 (ranges 82˜3630) LF/HF: 2.8 (ranges 1.1˜11.6)

A normal respiratory rate (RR) ranges from 12 to 20 per minute. If the RR exceeds 24 it is the Tachypnea. If the RR goes less than 10 it is Bradypnea.

These cardiovascular health indices based on ECG signal can be computed in the embedded software in microprocessor or computed by the applications of the smart phone, PC, and mobile devices. Some personal data can be used to compute individual cardiovascular health indices with ECG and PPG signal data. Normal values or ranges can be displayed with GUI, and auditory and visual warning signals can be provided if any abnormalities are detected.

FIG. 10 illustrates various elements of an exemplary system 1000 and related transmission features, such as associated with the mobile applications within the mobile environment(s), consistent with one or more aspects of the innovations herein. Referring to FIG. 10, a person 1002 is illustrated receiving thermal stimulation 1006 and providing ECG and/or PPG signals to a device 1004 or devices and/or system(s). A illustrative device, according to various embodiments herein, may be an integrated thermal stimulation and ECG/PPG measurement system or device 1004. The overall system 1000 may include such device 1004, another computing device 1010 such as a mobile device like a smartphone, one or more storage and/or display modules and/or elements (which may be integrated with system elements 1004 and/or 1010, or one or more separate or additional elements or devices), wired or wireless communication components in one or more of the system elements, a network 1030 such as a cloud network, and other participants 1040 such as doctors, hospitals, clinics, family members, or any other entity or person associated with such data and results.

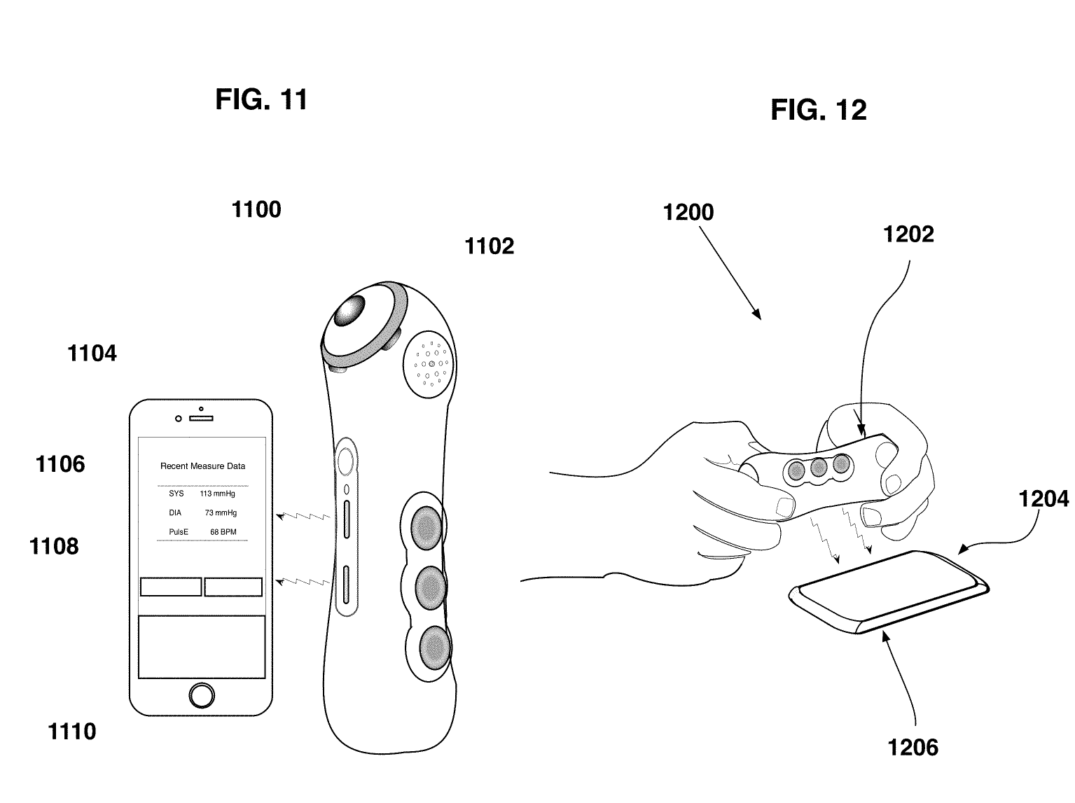

FIG. 11 illustrates an exemplary system 1100 including an application associated with a mobile device 1104 and involving mobile environment features, consistent with one or more aspects of the innovations herein. System 1100 includes mobile device 1104 that is configured to communicate with hypertension therapy device 1102. In some embodiments, the communication is performed wirelessly using Wi-Fi, Bluetooth, Zigbee, Infrared (IR), or other wireless communication standards. In an alternative embodiment, a proprietary wireless communication system may be utilized by the mobile device 1104 to communicate with a hypertension therapy device 1102.

The application associated with mobile device 1104 may be configured to facilitate the communication between the mobile device 1104 and hypertension therapy device 1102. In some embodiments, the application may provide a graphical user interface (GUI) that allows a user to configure the mobile device 1104 to collect blood pressure and/or other diagnostic information from the hypertension therapy device 1102. In one example, diagnostic information may be related to beat-to-beat variability of the measured heartbeat. Additionally, the application may provide a GUI that allows a user to determine the proper times to take the initial, intermediate, and final blood pressure measurements using the hypertension therapy device 1102. In some embodiments, the application may facilitate data logging functionality. The data logging functionality facilitates the monitoring of treatment efficacy over extended periods of time by storing information related to measurements performed.

In one embodiment, the application may operate in a diagnostic mode. The diagnostic mode provides a GUI that allows a user to operate the hypertension therapy device 1102 to collect blood pressure and/or other physiologic measurements. In this mode, the application may have a status window 1106 that displays the measurements taken. In one example, real time measurement data may be displayed, an average value of the measurement data may be displayed, and/or the maximum or minimum values of measurement data taken over a time period may be displayed. The application also includes an action window 1108 containing various buttons for performing physiologic measurements. In one example, the buttons may be used to begin, resume, pause, or end measurements. The application also includes a toolbar 1110 that allows users to select various modes of operation of the application. As an example, the toolbar 1110 may allow a user to toggle between a diagnostic mode, device status mode, data logging mode, or configuration mode. In an embodiment of application, the configuration mode may facilitate the pairing (e.g., establishing a communication link) between mobile device 1104 and hypertension therapy device 1102.

FIG. 12 illustrates an exemplary system 1200 including an application shown in use with a mobile device 1204 and involving mobile environment features, consistent with one or more aspects of the innovations herein. The mobile device 1104 is configured to communicate with hypertension therapy device 1102. The mobile device 1204 may be configured to run application 1206 which facilitates the diagnostic functionality of the hypertension therapy device 1102. As described above, the application 1206 may wirelessly transmit instructions to hypertension therapy device 1102 to collect blood pressure and/or other diagnostic information from the hypertension therapy device 1102 as illustrated in FIG. 12. Additionally, the application may allow a user to determine the proper times to take blood pressure measurements using the hypertension therapy device 1102. In some embodiments, the application may facilitate data logging functionality by storing and displaying measurement data.

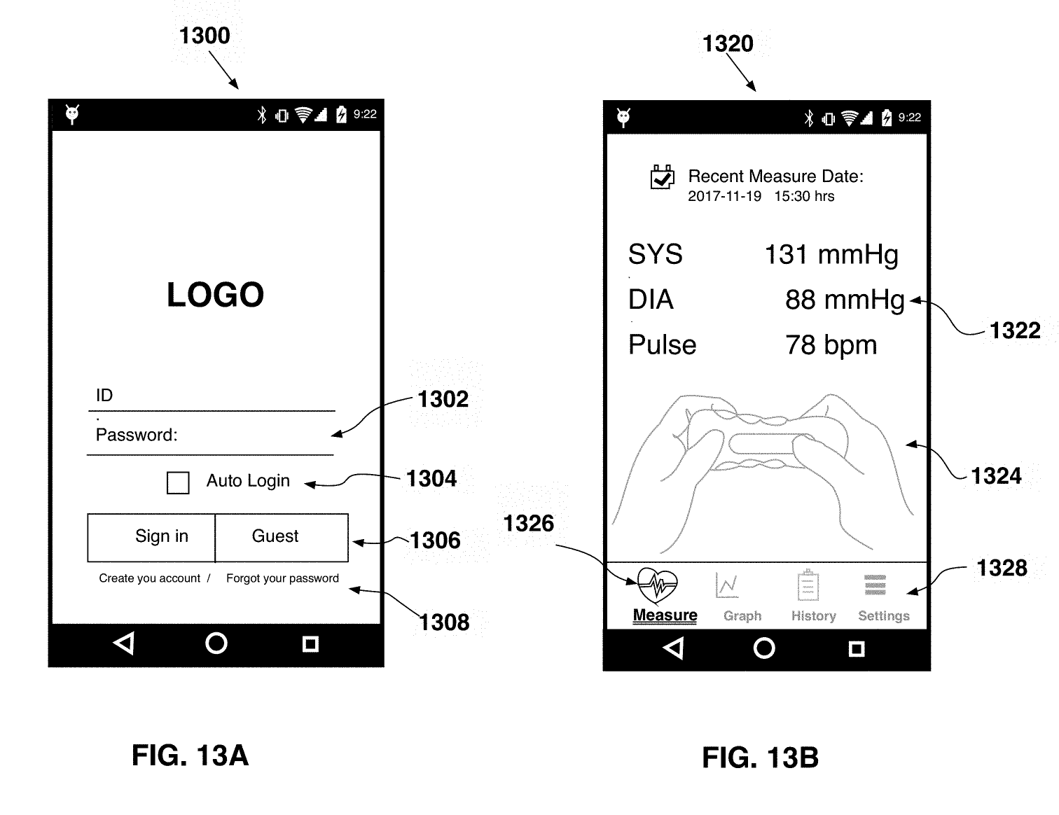

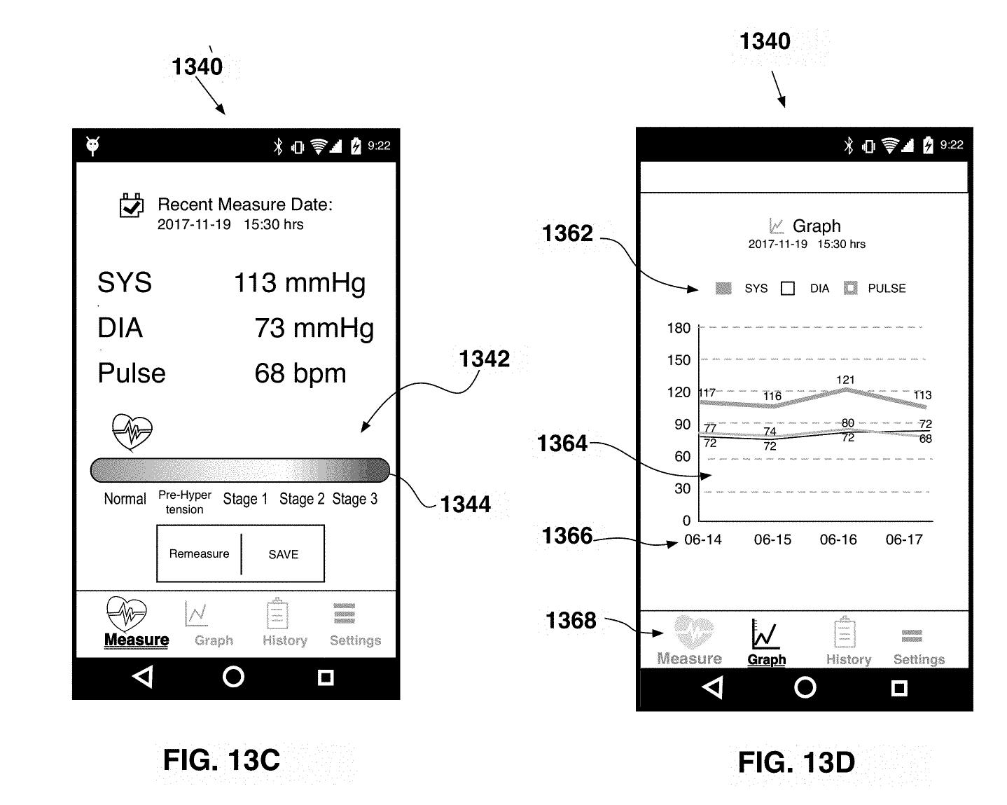

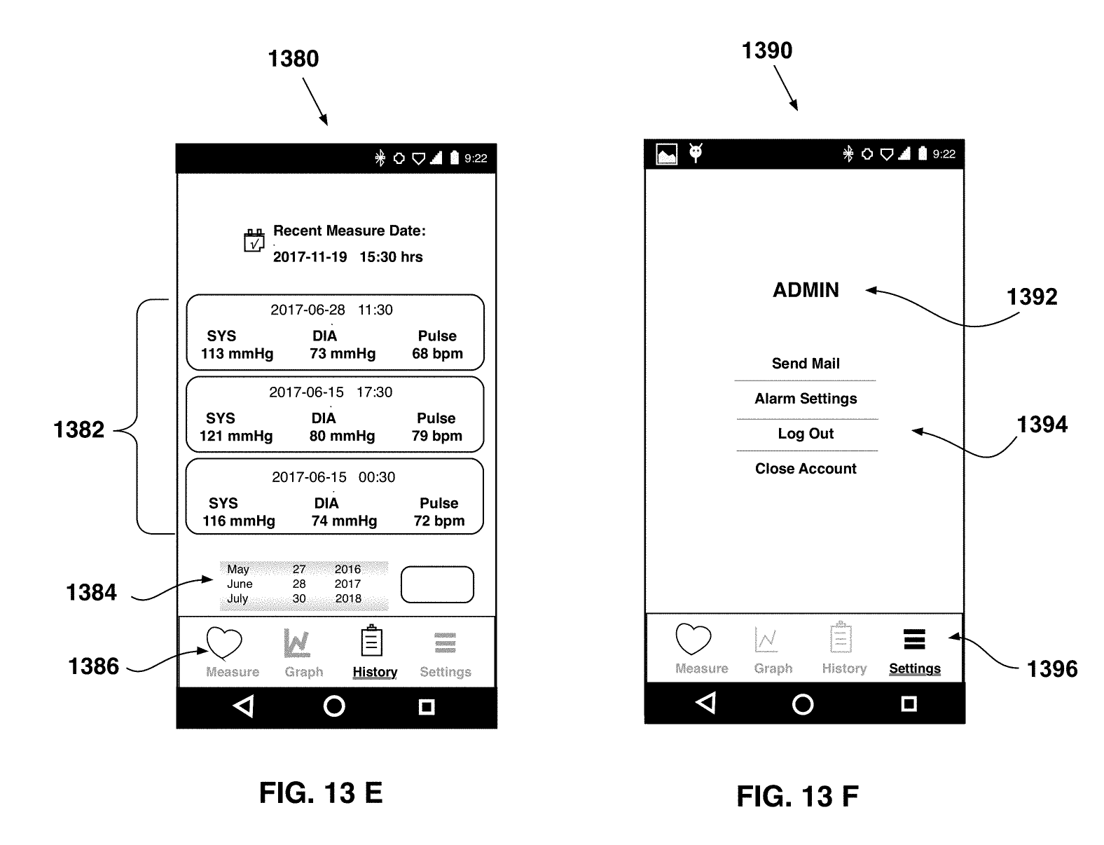

FIGS. 13A-13D illustrate exemplary innovations and associated mobile graphical user interface (GUI) aspects associated with representative implementations, including mobile environment innovations, consistent with one or more aspects of the innovations herein. FIG. 13A illustrates an exemplary interface for the login interface 1300 of the application. The login interface 1300 includes entry fields 1302 that allow users to enter account information. In an example, the user may input account ID and account password information. The login interface includes an auto login toggle button 1304 to save the account information in the application which will skip the login interface 1300 and directly bring forth the measurement interface 1320 when the application is opened. When the account information is created or verified with the sign in button in the button field 1306, the application will exit the login interface 1300 and allow the user to access the various other interfaces of the application.

FIG. 13B illustrates an exemplary interface for the measure interface 1320 of the application. The measure interface 1320 becomes accessible once the user account input in the previous login interface 1300 is verified or when the user selects a “measure” mode of operation from other sections of the application. The measure interface 1320 includes a mode toolbar 1328 that allows a user to toggle between various modes of operation. In one example, the verified user may access a “measure” mode, “graph” mode, “history” mode, and “setting” mode. Once selected, the application will display the interface associated with the selected mode. The mode toolbar 1328 may include an indicator 1326 to indicate the currently selected mode. For instance, the indicator 1326 in FIG. 13B indicates that a “measure” mode is selected and thus the corresponding measure interface 1320 is displayed.

The measure interface 1320 may include a measurement window 1322. The measurement window displays measurements taken by hypertension therapy device 1102 and transmitted to mobile device 1104. In the illustrated example, measurement data for “SYS,” “DIA,” and “PULSE” are displayed. In another example, the measurement data may be related to beat-to-beat variability of the measured heartbeat. The measurement data may correspond to the “measure date” indicated on the top of measure interface 1320. In other embodiments, the measurement data may be displayed in real time, as an average value over a period of time, and/or the maximum or minimum values of measurement data taken over a time period is displayed.

The measure interface 1320 may include a measuring indicator 1324. The measuring indicator 1312 displays the device condition while it is disconnected, connected, or measuring.

FIG. 13C illustrates an example interface for completed measurement page 1340. The measure interface 1320 switches to this new interface 1340 when the mobile device 1104 receives the measurement data from the hypertension therapy device 1102. The measure interface 1340 may include a diagnostic result indicator 1342. The diagnostic result indicator 1342 displays a visual representation that indicates the condition of the patient based upon the measurement data the collected and analyzed measurement data. In the illustrated example, the diagnostic result indicator 1312 indicates that the user condition is “NORMAL” based upon the collected and analyzed measurement data.

Alternative graphical representations may be provided if the measurement data indicates that the user's condition is not normal or requires additional attention. The measure interface 1340 may also include a action buttons 1344. The action buttons 1344 may allow the user to restart the measurement or save the measurement data.

FIG. 13D illustrates an exemplary interface for the graph interface 1360 of the application. The graph interface 1360 becomes accessible once the user information input in the previous login interface 1300 is verified or when the user selects a “graph” mode of operation from other sections of the application. The graph interface 1360 includes a mode toolbar 1368 that allows a user to toggle between various modes of operation. In one example, the verified user may access a “measure” mode, “graph” mode, “history” mode, and “setting” mode. Once selected, the application will display the interface associated with the selected mode.

The graph interface 1340 may include a graph key 1362. The graph key 1362 may indicate the data sets that are displayed on graph 1364. In the illustrated example, the graph key 1362 indicates that the “SYS,” “DIA,” and “PULSE” data sets are being displayed on graph 1364. In another example, the measurement data may be related to beat-to-beat variability of the measured heartbeat. The graph key 1362 may be color coded to match the color of the curve for each corresponding data set on graph 1346. In some embodiments, the x and y axis may have one or more scales to property fit the curves of each data set into one graph area. The graph interface 1360 may also contain a time frame toolbar 1366. The time frame toolbar 1366 may allow a user to select the desired time frame of data to be displayed on graph 1364. In the illustrated example, the user may toggle between a time frame of one day, one week, one month, or three months.

FIG. 13E illustrates an exemplary interface for the history interface 1380 of various display and processing features, consistent with one or more implementations of the innovations herein. The history interface 1380 becomes accessible once the user information input in the previous login interface 1300 is verified or when the user selects a “history” mode of operation from other sections of the application. The history interface 1380 includes a mode toolbar 1386 that allows a user to toggle between various modes of operation. In one example, the verified user may access a “measure” mode, a “graph” mode, a “history” mode, and a “setting” mode. Once selected, the application will display the interface associated with the selected mode. The mode toolbar 1386 may highlight or otherwise indicate the currently selected mode. For instance, the mode toolbar 1386 in FIG. 13E will indicate that a “history” mode is selected and thus the corresponding history interface 1380 is displayed.

The history interface 1380 may include a table 1382 that displays measurement data in table form. The table 1382 may include one or more data entries. The data entries may indicate the date and time that measurement data for the entry was taken and the measurement data recorded. In the illustrated example, table 1382 displays entries containing measurement data for “SYS,” “DIA,” and “PULSE.” The history interface 1380 may include a date picker 1384. The date picker 1384 may indicate the specific date of data set that is currently displayed on the history interface 1380. In the illustrated example, the date on the date picker 1382 indicates the date of measurement data that is being displayed in a table 1382.

In some embodiments, the history interface 1380 may include a data share system that allows users to transmit selected measurement data to their doctors or other healthcare providers. The data share system allows users to select data sets according to time frames, for example, selecting data collected over the last month or according to data type, for example, selecting data related to pulse or beat-to-beat heart rate variability. The data share system provides an integrated system for users to share their medical data including measurement data from the hypertension therapy device 1102. Using the system, healthcare providers may readily access the collected data, perform analysis on the data, and provide a diagnosis or treatment plan.

FIG. 13F illustrates an example of the setting interface 1400 of various display and processing features, consistent with one or more implementations of the innovations herein. The setting interface 1400 becomes accessible once the user selects the “setting” mode in the application. The setting interface 1400 may include a mode toolbar 1406 that allows a user to toggle between various modes of operation. In one example, the verified user may access a “measure” mode, a “graph” mode, a “history” mode, and a “setting” mode. Once selected, the application will display the interface associated with the selected mode. The mode toolbar 1406 may highlight or otherwise indicate the currently selected mode. For instance, the mode toolbar 1406 in FIG. 13F will indicate that a “setting” mode is selected and thus the corresponding history interface 1400 is displayed.

The setting interface 1400 may include an account indicator 1402. The account indicator 1402 may indicate the account name that is currently accessing the application. In one example, the setting interface 1400 may also include a menu table 1404 which allows the user to send data via email, set up push notifications, log out from the application and close the account.

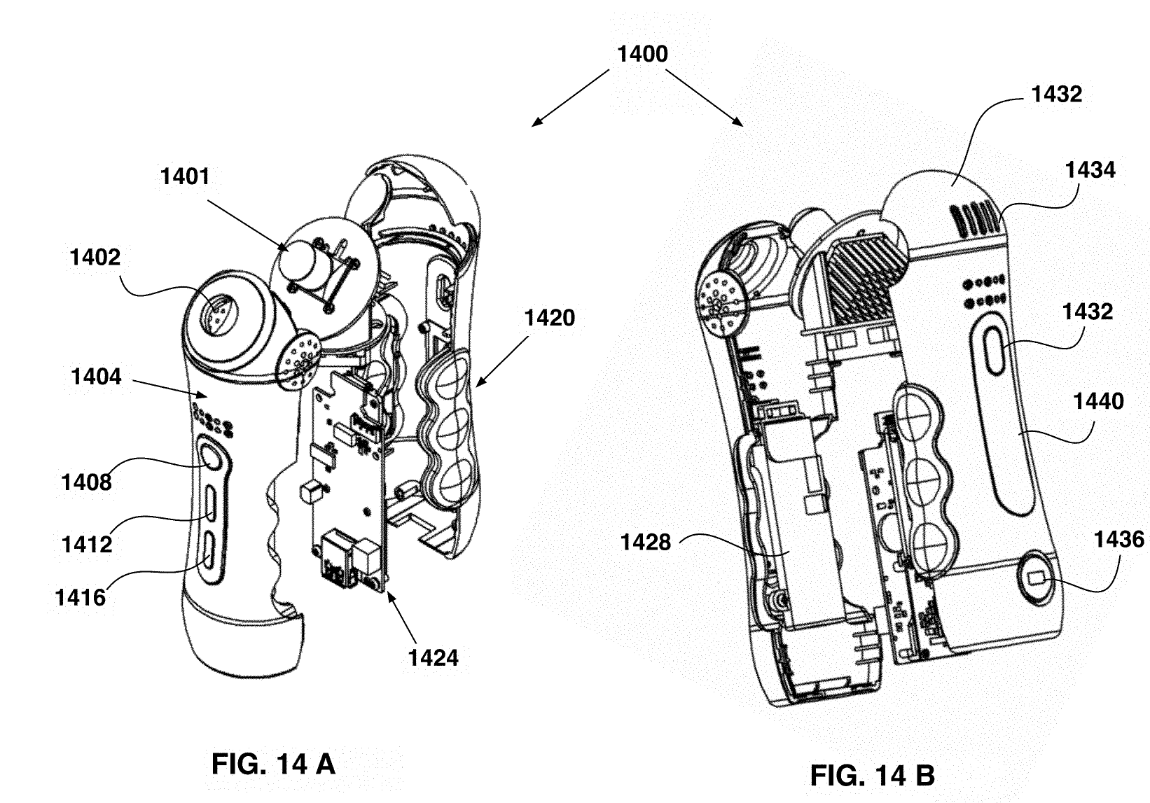

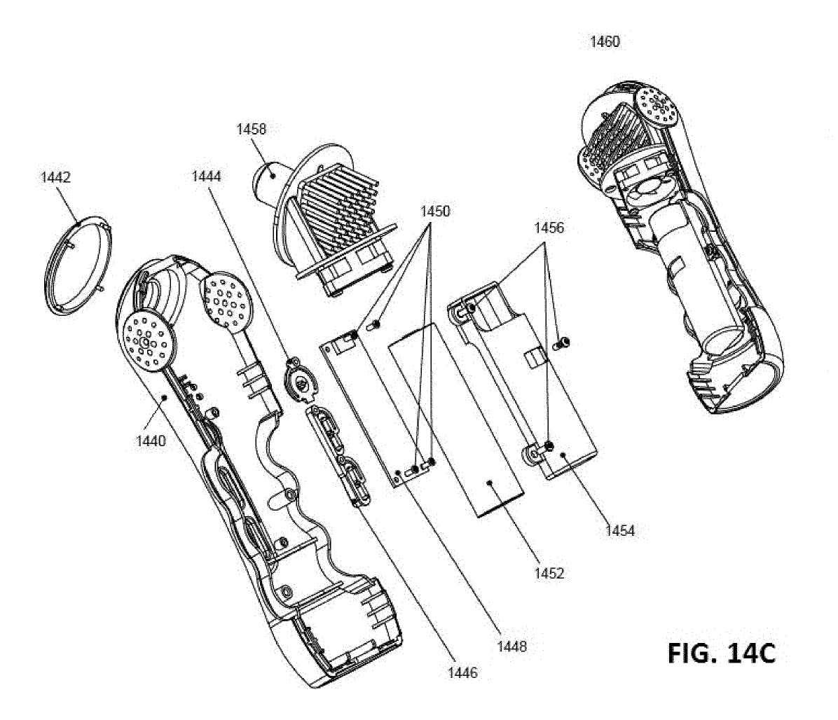

FIG. 14A-FIG. 14 B illustrate the thermal assembly 1401 that is used as point of treatment for carotid sinus. The front cover for the device 1404 may comprise ventilation holes. In one example, the power on switch 1408 may be located below the thermal therapy element 1402. Treatment switch 1412 may initiate the treatment when device is turned on. Biosensor switch 1416 may initiate the blood pressure measurement when device is turned on. Grip pad 1420 may provide for the secure handling of the device in the user's hand. PCB assembly 1424 may connect the main board, biosensor board, and display.

Battery 1428 may power the device. Back cover 1432 is shown in a posterior oblique view of the device with ventilation holes 1434. The ECG electrode 1432 may be used as the indifferent electrode for biosensor mode sensing the electrocardiogram. The PPG electrode 1436 may be used for acquiring the spectroscopic data for determining the blood pressure in combination with the ECG data in the biosensor mode. LCD display cover 1440 is a display that may provide the data and instructions for viewing by the user. The display changes with each of the different modes showing blood pressure measurements and other data depending upon the operating mode.

FIG. 14C provides an exemplary exploded view of the various display and processing features, consistent with one or more implementations of the innovations herein. Front cover 1440 is shown. Deco-ring 1442 is also shown and may be used in some embodiments of the present invention. In some embodiments, the present invention has an interval view 1460. In some embodiments, power-key 1444 is located next to key-block 1446. And Switch-board 1448 may additionally be present in some embodiments. Tapping Screw 1450 may be used on the switch-board 1448 and a battery assembly 1452 may also be present. A battery holder 1454 may additionally be present and tapping screw 1456 may be used on the battery holder 1454. Thermoelectric module 1458 may also be a component of the present invention.

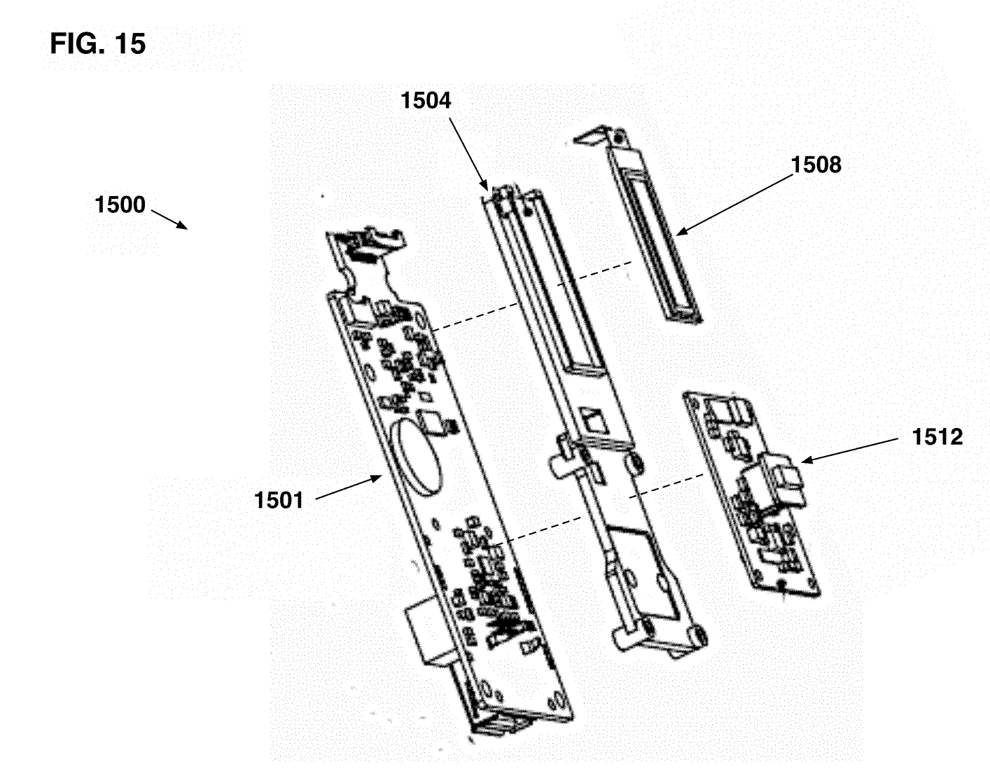

FIG. 15 illustrates an exploded view of the internal electronic components. PCB 1501 may connect to the thermal module, switches, display, biosensor board, and battery when assembled. PCB assembly fixture 1504 may support the main PCB, display, and biosensor. LCD Display 1508 may be used to display device status. Biosensor board 1512 may be placed below the PPG electrode on the cover. The Biosensor board 1512 may be used to calculate the blood pressure of the patient.

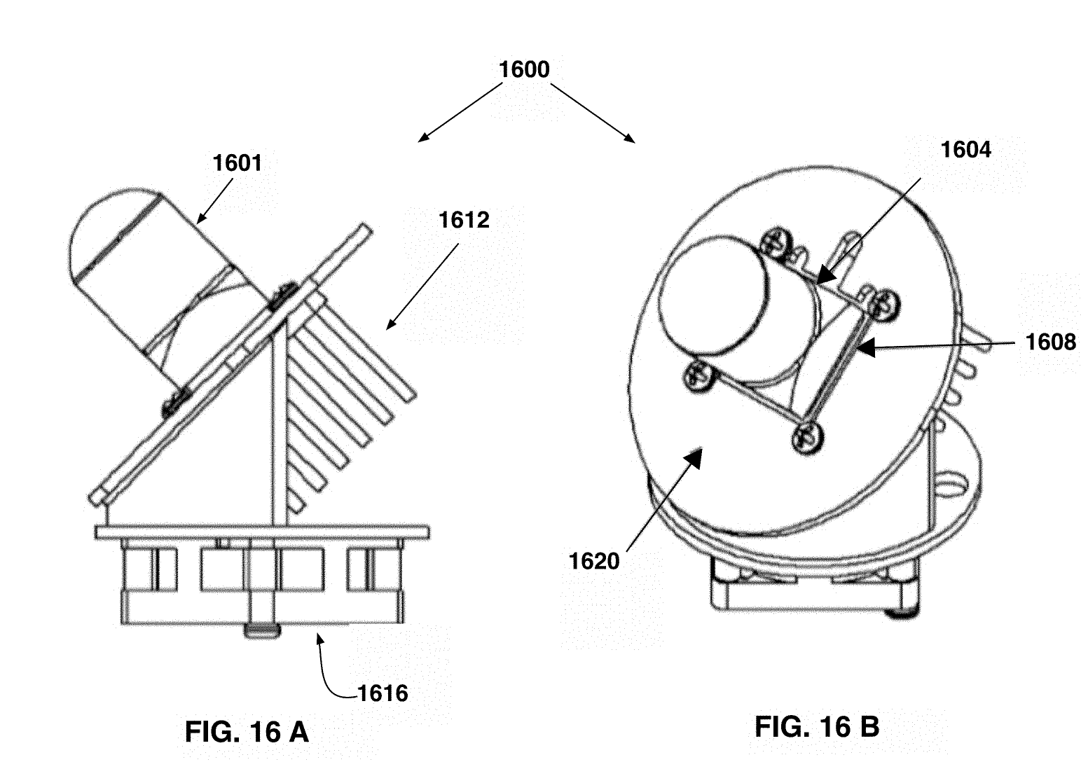

FIG. 16A is a lateral view and FIG. 16B an oblique view of the Peltier element metal tip 1601 that is used to apply hypothermic energy to the patient's carotid sinus. The NTC thermistor 1604 inside of the tip used to monitor and control tip temperature. Heat sink 1612 is used to dissipate heat from the hot side of TEC module. TEC module 1608 may serve as a source for cold temperature to the tip. Fan 1616 may be used to expel heat from around the thermal assembly. Supporting case structure 1620 is the foundation for the fan and heat sink, which are whereupon screwed into place.

FIG. 17A-17B illustrate layout positions of bio-sensor (ECG/PPG) and electrodes 1701-1704, 1708-1712 embedded in the steering wheels of an automobile 1700. In some embodiments, bio-sensor (ECG/PPG) and electrodes are embedded in the steering wheels of a jet, a tank, a boat, a truck, or other types of mode of transportation. By placing a person's thumbs on the sensors on the steering wheel before and/or during and/or after driving, the automobile's ECG/PPG signals will provide cardiovascular health indices and measure cardiovascular health data from drivers' and/or passengers' fingers. FIG. 17A illustrates the placement of the biosensors 1701 and 1704, in the support structure of the steering wheel and FIG. 17B shows the biosensors 1708 and 1712 implemented in the outer rim of the steering wheel along the circumference. This is an efficient and comfortable interface that measures, records, and analyses. FIG. 17C illustrates the placement of the physiologic readings monitor 1720 situated in the automobile's gage panel 1716 along with the car's tachometer and speedometer. In some embodiments, the readings monitor 1720 may be placed in other available displays in the car, jet, tank, boat, truck or other types of mode of transportation.

FIG. 18A-18B illustrate layouts of the cardiovascular indices with data such as human temperature, heart rate, blood pressure data and other physiological states in the automobile control display and/or main display panels 1811. In some embodiments, the layouts may be displayed in cars, jets, tanks, boats, trucks or other types of mode of transportation.

FIG. 18A is an example of a layout 1801 of the cardiovascular indices presented to the driver/user with data such as human temperature, heart rate, blood pressure data and other physiological states in the automobile control display and/or main display panels 1720. FIG. 18B is an example of the user interface in a GUI equipped vehicle showing the presentation of the many apps that may be available to the occupants of the vehicle with the BP monitoring app selection icon 1808 displayed in a position on the control on the screen. In some embodiments, the display of physiological states and apps may be displayed in cars, jets, tanks, boats, trucks or other types of mode of transportation.

### Peltier Effect

The thermoelectric module 1458 operates by the peltier effect. The peltier effect, as described previously, is the result of a temperature difference occurring from an electrical power being run between two electrodes of dissimilar materials. This principle relies on the idea that a heat current accompanies electrical current.

Typical thermoelectric modules 1458 consist of two or more n and p-type doped semiconductor materials mounted between two ceramic substrates. The ceramic substrates work to hold the overall structure together. These semiconductors are connected electrically in series and thermally in parallel. As the current is run through this junction, heat will move through the module from one side to the other by forced convection. One side of the module will absorb all the heat within the system and the other side will release it. This produces the lower and higher temperature sides respectively. The temperature difference observed in the thermoelectric module 1458 is due to the flow of electrons from the conductor that has less bound electrons to the one that has highly bound electrons. However, as an electric current continues to pass through the module, the heat that is released will start to exceed the heat absorbed. This will cause both sides to reach relatively hot temperatures and be insufficient to serve as a cooling source. Therefore, the temperature difference within the thermoelectric module 1458 must be controlled so that the lower temperature side can be used as a source for cooling.

In order to control the temperature of the lower temperature side, the thermal mass of the higher temperature side must be increased. Thus, a heat sink and cooling mechanism are used in these cooling applications as means of increasing the thermal mass of the higher temperature side of the thermoelectric module 1458. By increasing the thermal mass of the higher temperature side, it would be possible to control the lower temperature side. The lower temperature side of the thermoelectric module 1458 is in contact with the hypertension treatment device tip to act as a cold source. This allows the tip to attain its cold temperature and carry out the hypertension treatment. However, as the hypertension treatment device (and ultimately the thermoelectric module 1458) ceases operation, current stops passing through the thermoelectric module 1458 and there will no longer be a maintenance of temperature difference between the two sides. This will cause a change of temperature to be experienced, where the two sides will reach a neutral temperature. Due to the higher thermal mass of the higher temperature side as compared to the lower temperature side, this neutral temperature will be higher than that of room temperature. Thus, there would be a transfer of heat occurring from one side of the module to the other until a uniform temperature is experienced and thermal equilibrium is reached. As a result, the tip temperature will rise to be higher than room temperature. This increased temperature, however, is not ideal for continuous and consecutive hypertension treatment beyond powering of the thermoelectric module 1458. Therefore, there is the need to provide additional cooling to maintain optimal temperature conditions for effective continuous and consecutive hypertension treatment device use.

### Cooling Mechanism Method

A method disclosed herein can include extended powering of the cooling mechanism to maintain optimal tip temperature for consecutive use as shown in FIG. 1. The method can have communication between the cooling mechanism and the hypertension treatment device timer. The method can have a switch activate once the hypertension treatment device timer ends to allow for activation of the cooling mechanism for an extended duration of time. The method can have a preset timer activated alongside the switch to determine when the switch and subsequently the cooling mechanism be turned off. The method can have a temperature sensor that senses the temperature of the hotter side and detects how long the cooling mechanism should be powered beyond removal of power from the thermoelectric module 1458.

Appendix A shows how the present bio-sensors innovations may capture physiologic parameters from people, e.g., to send to the cloud. Such bio sensor features can analyze electocardiographic activity in combination with spectrographic (ECG and PPG signal) readings of the variation vascular capillary and analysis to yield improved test data.

1. (canceled)

2. A hypertension treatment device that measures one or more cardiovascular health indices and administers hypertension treatment, the treatment device comprising:
a handheld instrument including a treatment head coupled to a thermal assembly configured to adjust temperature of the head; and
an electrocardiogram (ECG) sensing module and a photoplethysmography (PPG) sensor configured to measure a plurality of the cardiovascular health indices;
wherein the health device is configured to administer hypertension treatment based on a plurality of the measured cardiovascular health indices.

3. The treatment device of claim 2, wherein administering the hypertension treatment comprises adjusting the temperature of the treatment head.

4. A hypertension treatment device that measures one or more cardiovascular health indices and administers hypertension treatment, the treatment device comprising:
an electrocardiogram (ECG) sensing module or electrode configured to measure at least a first cardiovascular health index;
at least one photoplethysmography (PPG) sensor configured to measure at least a second cardiovascular health index, wherein the PPG sensor is located within an outer boundary of the ECG electrode.

5. The treatment device of claim 4, wherein the PPG sensor is located at or near the center of the ECG electrode.

6. The treatment device of claim 4, wherein the PPG sensor further comprises a light-emitting diode or an optical source and a photosensor or an optical sensor.

7. The treatment device of claim 2 wherein the electrocardiogram (ECG) sensing module comprises at least one ECG electrode.

8. The treatment device of claim 2 wherein the electrocardiogram (ECG) sensing module comprises two ECG electrodes, each positioned on the instrument and shaped to engage a digit (thumb or finger) of the user's opposite hands.

9.-13. (canceled)

14. A hypertension treatment device that measures one or more cardiovascular health indices and administers hypertension treatment, the treatment device comprising:
an elongated body comprising a first end, a second end, a back middle portion, and a front middle portion;
a treatment head located at the first end of the body coupled to a thermal assembly, wherein the thermal assembly is disposed within the treatment device and further comprises:
a thermistor or first sub-component disposed within the treatment device configured to monitor and control the temperature of the treatment head; and
a thermoelectric sub-component configured to adjust the temperature of the treatment head;
at least one control interface on the body;
at least one electrocardiogram (ECG) sensing module and at least one photoplethysmography (PPG) sensor located on the treatment device to receive vital sign measurements;
a wireless communication circuit configured to transmit data to a receiver and receive data or commands from a remote device;
a memory circuit configured to store first information captured by the treatment device and/or second information received from a remote device;
a signal processor coupled to the wireless communication circuit;
a visual display configured to display vital statistics; and
biosensor circuitry disposed within the treatment device coupled to the at least one sensors.

15. The treatment device of claim 14, wherein the remote device includes the receiver.

16. The treatment device of claim 14, wherein the one or more cardiovascular health indices includes one or more of blood pressure, heart rate, body temperature, and respiratory rate.

17. The treatment device of claim 14, further comprising a plurality of ventilation holes for dissipating heat generated from within the treatment device.

18. The treatment device of claim 14, further comprising a heat sink configured to dissipate heat from the hot side of the thermoelectric sub-component.

19. The treatment device of claim 14, further comprising an authentication circuit configured to verify an authorized user.

20. The treatment device of claim 14, wherein the signal processor is further coupled to one or more of the ECG or PPG sensors.

21. The treatment device of claim 14, wherein the PPG sensor includes a photo sensor and an LED.

22. The treatment device of claim 14, wherein the ECG sensor includes the PPG sensor as an integrated assembly.

23. The treatment device of claim 14, wherein the ECG sensor comprises a recessed portion of the body forming a concave shape, wherein the PPG sensor is located within the recessed portion.

24. The treatment device of claim 23, wherein the PPG sensor is disposed within the ECG sensor.

25. The treatment device of claim 14, wherein the treatment head comprises a metal tip or a tip constructed of other, thermally-conductive material.

26. The treatment device of claim 14, wherein the treatment head comprises a curved or convex geometry.

27.-56. (canceled)
